SEQUENCE LISTING (53 sequences)

SEQ ID NO: 1 - protein, 8 aa, synthetic construct
  REGION 1..8: note = Example 1.1 peptide 1
ALWDKSNL

SEQ ID NO: 2 - protein, 9 aa, synthetic construct
  REGION 1..9: note = Example 1.1 peptide 2
SQFSSIWKK

SEQ ID NO: 3 - protein, 9 aa, synthetic construct
  REGION 1..9: note = Example 1.1 peptide 3
AEAHSVSLI

SEQ ID NO: 4 - protein, 8 aa, synthetic construct
  REGION 1..8: note = Example 1.1 peptide 4
YQHNDLQL

SEQ ID NO: 5 - protein, 10 aa, synthetic construct
  REGION 1..10: note = Example 1.1 peptide 5
RLLPLTKSPI

SEQ ID NO: 6 - protein, 17 aa, synthetic construct
  REGION 1..17: note = Example 1.1 peptide 6
NGLTESQFSSIWKKVSD

SEQ ID NO: 7 - protein, 18 aa, synthetic construct
  REGION 1..18: note = Example 1.1 peptide 7
RLLPLTKSPIESTTEPPA

SEQ ID NO: 8 - protein, 19 aa, synthetic construct
  REGION 1..19: note = Example 1.1 peptide 8
ELIGPAEAHSVSLIDTFPE

SEQ ID NO: 9 - protein, 17 aa, synthetic construct
  REGION 1..17: note = Example 1.1 peptide 9
LSSSHSYQHNDLQLRST

SEQ ID NO: 10 - protein, 16 aa, synthetic construct
  REGION 1..16: note = Example 1.1 peptide 10
NECEKALWDKSNLTKH

SEQ ID NO: 11 - protein, 10 aa, synthetic construct
  REGION 1..10: note = Example 1.2 peptide 1
YKIYNLYAER

SEQ ID NO: 12 - protein, 12 aa, synthetic construct
  REGION 1..12: note = Example 1.2 peptide 2
LYAERHYDTAKF

SEQ ID NO: 13 - protein, 9 aa, synthetic construct
  REGION 1..9: note = Example 1.2 peptide 5
CLNKGHYIK

SEQ ID NO: 14 - protein, 19 aa, synthetic construct
  REGION 1..19: note = Example 1.2 peptide 7
NHYKIYNLYAERHYDTAKF

SEQ ID NO: 15 - protein, 17 aa, synthetic construct
  REGION 1..17: note = Example 1.2 peptide 10
FHMVVEVPHWSNAKMEI

SEQ ID NO: 16 - protein, 17 aa, synthetic construct
  REGION 1..17: note = Example 1.2 peptide 12
EGARMWINNMKMRSFSP

SEQ ID NO: 17 - protein, 17 aa, synthetic construct
  REGION 1..17: note = Example 1.2 peptide 14
LPSDQLELIATGVFAKP

SEQ ID NO: 18 - protein, 9 aa, synthetic construct
  REGION 1..9: note = Example 1.2 peptide 3
IMVAIYVNR

SEQ ID NO: 19 - protein, 10 aa, synthetic construct
  REGION 1..10: note = Example 1.2 peptide 4
RMWINNMKMR

SEQ ID NO: 20 - protein, 10 aa, synthetic construct
  REGION 1..10: note = Example 1.2 peptide 6
NDSQRKFNSR

SEQ ID NO: 21 - protein, 17 aa, synthetic construct
  REGION 1..17: note = Example 1.2 peptide 8
LKTHISKGMLGKFTVPM

SEQ ID NO: 22 - protein, 17 aa, synthetic construct
  REGION 1..17: note = Example 1.2 peptide 9
EEEPGGAAMREVYEEAG

SEQ ID NO: 23 - protein, 17 aa, synthetic construct
  REGION 1..17: note = Example 1.2 peptide 13
SLSNDSQRKFNSRPGTG

SEQ ID NO: 24 - protein, 9 aa, synthetic construct
  REGION 1..9: note = Example 1.3 peptide 1
LLGRNSFEL

SEQ ID NO: 25 - protein, 9 aa, synthetic construct
  REGION 1..9: note = Example 1.3 peptide 2
SALETVEVL

SEQ ID NO: 26 - protein, 9 aa, synthetic construct
  REGION 1..9: note = Example 1.3 peptide 3
YLAARNILV

SEQ ID NO: 27 - protein, 9 aa, synthetic construct
  REGION 1..9: note = Example 1.3 peptide 4
FEVDQRQGL

SEQ ID NO: 28 - protein, 11 aa, synthetic construct
  REGION 1..11: note = Example 1.3 peptide 5
YEFGQALSTGW

SEQ ID NO: 29 - protein, 9 aa, synthetic construct
  REGION 1..9: note = Example 1.3 peptide 6
MVHRLVKLF

SEQ ID NO: 30 - protein, 14 aa, synthetic construct
  REGION 1..14: note = Example 1.3 peptide 7
NLLGRNSFELRVCA

SEQ ID NO: 31 - protein, 17 aa, synthetic construct
  REGION 1..17: note = Example 1.3 peptide 9
EHKSGVLDASTIATILR

SEQ ID NO: 32 - protein, 17 aa, synthetic construct
  REGION 1..17: note = Example 1.3 peptide 11
RRHAFRRQDSSTFSYYG

SEQ ID NO: 33 - protein, 17 aa, synthetic construct
  REGION 1..17: note = Example 1.3 peptide 12
DFNLEKSRIKELYSLNE

SEQ ID NO: 34 - protein, 9 aa, synthetic construct
  REGION 1..9: note = Example 1.4 peptide 1
YLDDRNTFQ

SEQ ID NO: 35 - protein, 9 aa, synthetic construct
  REGION 1..9: note = Example 1.4 peptide 2
PMHGDLDLV

SEQ ID NO: 36 - protein, 8 aa, synthetic construct
  REGION 1..8: note = Example 1.4 peptide 3
SLQNTPVL

SEQ ID NO: 37 - protein, 11 aa, synthetic construct
  REGION 1..11: note = Example 1.4 peptide 4
VTQDTQNELKK

SEQ ID NO: 38 - protein, 10 aa, synthetic construct
  REGION 1..10: note = Example 1.4 peptide 5
AEDQPMHGDL

SEQ ID NO: 39 - protein, 9 aa, synthetic construct
  REGION 1..9: note = Example 1.4 peptide 6
TSDDILQKI

SEQ ID NO: 40 - protein, 10 aa, synthetic construct
  REGION 1..10: note = Example 1.4 peptide 7
STHLGEKSCK

SEQ ID NO: 41 - protein, 17 aa, synthetic construct
  REGION 1..17: note = Example 1.4 peptide 8
YLDDRNTFQHSVVVPYE

SEQ ID NO: 42 - protein, 17 aa, synthetic construct
  REGION 1..17: note = Example 1.4 peptide 9
NVIRDAVTDTEHAKRKT

SEQ ID NO: 43 - protein, 18 aa, synthetic construct
  REGION 1..18: note = Example 1.4 peptide 10
HLAGYRQHDAHEFLIAIL

SEQ ID NO: 44 - protein, 9 aa, synthetic construct
  REGION 1..9: note = Example 1.5 peptide 1
LSLLQVQLY

SEQ ID NO: 45 - protein, 9 aa, synthetic construct
  REGION 1..9: note = Example 1.5 peptide 2
ITTYKKKLR

SEQ ID NO: 46 - protein, 9 aa, synthetic construct
  REGION 1..9: note = Example 1.5 peptide 3
LTRSRSEGR

SEQ ID NO: 47 - protein, 10 aa, synthetic construct
  REGION 1..10: note = Example 1.5 peptide 4
EFHVKTEQLR

SEQ ID NO: 48 - protein, 10 aa, synthetic construct
  REGION 1..10: note = Example 1.5 peptide 5
LSCPTSFRSY

SEQ ID NO: 49 - protein, 9 aa, synthetic construct
  REGION 1..9: note = Example 1.5 peptide 6
FGFHFVLVL

SEQ ID NO: 50 - protein, 16 aa, synthetic construct
  REGION 1..16: note = Example 1.5 peptide 7
NRSKSKNEKKILGQKK

SEQ ID NO: 51 - protein, 17 aa, synthetic construct
  REGION 1..17: note = Example 1.5 peptide 8
EEKKDDENVPMETEETH

SEQ ID NO: 52 - protein, 17 aa, synthetic construct
  REGION 1..17: note = Example 1.5 peptide 9
SRFFADSGTDPKGEPGS

SEQ ID NO: 53 - protein, 17 aa, synthetic construct
  REGION 1..17: note = Example 1.5 peptide 10
KFLISNEKQIFSENSLK

(19) United States
(12) Patent Application Publication
(10) Pub. No.: US 2025/0161421 A1
(43) Pub. Date: May 22, 2025

(54) CO-VACCINATION WITH CD4 AND CD8 ANTIGENS

(71) Applicant: CECAVA GMBH & CO. KG, Tübingen (DE)

(72) Inventors: Henning Zelba, Dusslingen (DE); Saskia Biskup, Tübingen (DE); Dirk Hadaschik, Köln (DE); Oliver Bartsch, Tübingen (DE); Magdalena Feldhahn, Kusterdingen (DE); Christina Kyzirakos-Feger, Tübingen (DE); Simone Kayser, Tübingen (DE)

(21) Appl. No.: 18/835,168

(22) PCT Filed: Feb. 3, 2023
(86) PCT No.: PCT/EP2023/052687

§ 371 Date: Aug. 1, 2024

(30) Foreign Application Priority Data
Feb. 3, 2022 (EP) 22155088.2

(51) Int. Cl. A61K 39/00; A61P 37/04

(52) U.S. Cl. CPC A61K 39/001114; A61K 39/001111; A61P 37/04

(57) ABSTRACT

The present invention relates to a vaccine composition comprising a first peptide being an MHC I antigen and a second peptide being an MHC II antigen, wherein the sequence of the first peptide is comprised in the sequence of the second peptide. The present invention further relates to a kit of parts comprising the same. Instant compositions and kits are particularly useful in the methods of inducing or increasing an immune response in a subject. Instant compositions are further useful in the methods of treatment or prevention of a disease or a disorder in a subject, in particular an infectious disease or a cancer disease.

### FIELD OF THE INVENTION

The present invention relates to a vaccine composition comprising a first peptide being an MHC I antigen and a second peptide being an MHC II antigen, wherein the sequence of the first peptide is comprised in the sequence of the second peptide. The present invention further relates to a kit of parts comprising the same. Instant compositions and kits are particularly useful in the methods of inducing or increasing an immune response in a subject. Instant compositions are further useful in the methods of treatment or prevention of a disease or a disorder in a subject, in particular an infectious disease or a cancer disease.

### BACKGROUND OF THE INVENTION

Tumour cells always acquire genome alterations (mutations) and/or changes in gene expression in the course of their development and consequently in proteome composition when compared to normal cells. These changes can be recognized by the immune system as foreign, which can lead to the destruction of the tumour cells by, for example, immune cells like T-cells. Immune therapies like immune checkpoint inhibition very efficiently unleash the suppression of T-cells in the tumour, which can subsequently recognize and kill the tumour cells. However, many patients do not benefit from immune checkpoint inhibitor therapy. To this end, vaccination against antigens of tumour origin in order to activate the immune system to combat cancer could be important. However, most efforts in the last 30 years have been practically unsuccessful. With today's knowledge, however, there is new hope.

The principal goal of therapeutic and prophylactic vaccines is the induction and/or enhancement of an antigen-specific cellular (and humoral) immune response. Within the so-called adaptive immune system, T-cells and antigen-presenting cells (APC) play a key role in this process.

T-cells, which recognize endogenous antigens, are eliminated in the thymus during maturation. Thus, theoretically only those T-cells should leave the thymus which can recognize foreign or altered antigens, i.e. pathogen antigens or antigens comprising mutations. Such naïve T-cells get activated and expand in numbers upon recognition of their antigens which were taken up and presented by antigen presenting cells (APC) patrolling the body. The recognition of the antigen is mediated by the T-cell receptor (TCR), a protein complex on the surface of T-cells. The aim of a vaccination is therefore i) to present individual naïve T-cells with their corresponding antigen in order to activate and expand them and/or ii) to reactivate them if memory T-cells already exist from a previous exposure to the antigen.

The antigen consists of a protein-derived antigenic peptide (epitope) presented on the major histocompatibility complex (MHC). The MHC I is present on all somatic cells and has the function to bind to 8 to 13 amino acids long epitopes derived from mainly endogenous proteins which are proteolytically cleaved intracellularly and to present such peptides on the cell surface to cytotoxic T-cells (CD8+ T-cells). Such T-cells bind to MHC I with the help of their CD8 receptor which supports antigen scanning and recognition by the TCR. APC can present such short antigens via MHC I together with co-activating receptors. CD8+ T cells recognizing the APC presented antigen get activated, expand in numbers and express cytotoxic proteins stored in intracellular granules. If such patrolling activated cytotoxic T cells later recognize their specific antigen on other somatic cells, they may release cytotoxic proteins to kill such cells. By this mechanism T cells can detect and kill cells which present foreign antigens derived from intracellular pathogens like viruses or tumour mutations.

MHC II molecules are exclusively expressed on antigen-presenting cells (APC) such as B cells, monocytes, macrophages and dendritic cells which can phagocytize exogenous material. MHC II binds and presents peptide epitopes that are typically in a range of 14 to 35 amino acid residues, peaking at 15 residues. Said epitopes are predominantly of extracellular origin (e.g. from taken up pathogens). T helper cells (CD4+ T cells) bind to MHC II with the help of the CD4 receptor which supports antigen scanning and recognition by the TCR. Upon antigen recognition T helper cells get activated and via secretion of cytokines may support activation and modulation of other immune cells like cytotoxic T cells.

Normally, every cell in the body presents via MHC I molecules a plurality of peptides that have originated from thousands of cellular proteins. These peptides are formed in the cell through enzymatic degradation of cellular proteins as part of normal metabolism. While most of the cellular proteins are broken down to single amino acid residues in the process of proteostasis, a small subset remains in form of peptides that may be loaded onto the MHC molecules in the endoplasmic reticulum and brought to the cell surface. The complex of MHC molecule and peptide can then be recognized by T-cells. If the presented peptides are exclusively derived from normal (unaltered) cellular proteins, these cells are recognized as self or normal and no T-cell response is elicited. In other words, if there are no foreign peptides presented on MHC molecules, no T-cells able to recognize these peptides should be present due to negative selection in the thymus. However, for example in the case of a virus infection, the MHC-presented peptides include also some foreign peptides originating from the virus proteins, which can then be recognized by the corresponding T-cells. As the consequence the T-cell kills the infected cell.

In case of cancer, the situation is similar. T-cells can recognize for example, the so-called mutated neoantigens, which are peptides that result from a tumour-specific mutation and therefore have a different amino acid sequence than the corresponding normal protein. It is assumed that such neoantigens are recognized by the immune checkpoint inhibitor (ICI)-mediated T-cell response, since ICI response rate correlates with the number of mutations at the DNA level. Furthermore, tumour cells present also non-mutated tumour-specific peptides that are not found in normal cells. Such unmutated tumour-associated antigens (TAA) may be derived from genes which are highly upregulated mainly in tumour cells or which are from genes aberrantly expressed in the tumour such as cancer testis antigens (CTA usually only expressed in germ cells and tumour cells) or oncofetal antigens (typically only expressed in fetal tissues and in cancerous cells). T-cells may hence also recognize tumour cells presenting peptides derived from such unmutated TAA.

A special case are oncoviral antigens derived from tumourigenic transforming viruses, which can also be detected by T-cells on the surface of infected tumour cells.

Tumour-specific peptides, whether or not comprising tumour-specific (somatic) mutations, would thus be ideal candidates for therapeutic vaccination. However, there are two major obstacles that have prevented significant success so far. These peptides are different in each person, both because of the diversity of different malignant cells differing largely in somatic mutations and/or aberrantly expressed genes and because of the pronounced polymorphism and differing peptide specificity of the MHC molecules. Furthermore, in clinical studies, it has so far been practically unsuccessful to induce clinically effective T-cell responses against such peptides in patients by vaccination.

There is thus an urgent need for new vaccine compositions and, in particular, new cancer vaccines with improved ability of inducing and/or increasing an immune response upon administration to a subject.

Document WO 2017/097699 discloses sequences of specific peptides and their combination for use in immunotherapy against various cancers.

Document EP 2′111′867 discloses certain formulations of tumour-associated peptides binding to human leukocyte antigen (HLA) class I or II molecules for vaccines.

Document EP 3′604′325 discloses certain cancer antigenic peptides from Wilms Tumour Protein WT1 and peptide conjugate bodies containing the same.

Document Lohia Neha et al. (“Conserved Peptides Contaning Overlapping CD4+ and CD8+ T-Cell Epitopes in the H1N1 Influenza Virus: An Immunoinformatics Approach”, Viral Immunology, vol 27, no. 5, 1 Jun. 2014, pages 225-234) discloses the identification of CD4+ and CD8+ T-cell epitopes from the hemagglutinin (HA) and neuraminidase (NA) protein of influenza virus which led to peptides containing overlapping CD4+ and CD8+ T-cell epitopes and puts forward that such conserved peptides containing several epitopes would be the ideal candidate for an universal vaccine.

Document WO 2020/043805 discloses a method for ranking and selecting tumour-specific neoantigens comprising the preparation of a vaccine with up to 20 personalized peptides.

Halpert et al. (The FASEB Journal. 2020; 34:8082-8101) describe certain pathogen-associated molecular patterns (PAMP) requiring peptide epitopes with stretches of sequence identity bound to both MHC I and MHC II of DC leading to the induction of TH1 immune polarization and activation of the cellular immune response.

### SUMMARY OF THE INVENTION

Previous vaccination strategies apply either MHC I antigens to activate cytotoxic CD8+ T-cells or MHC II antigens to activate CD4+ T helper cells against a specific target antigen (e.g. a tumour mutation-derived neoantigen, unmutated TAA or pathogen antigen). Sometimes only long MHC II antigens are used containing a predicted MHC I epitope in order to activate both T-cell populations (CD4+ and CD8+ T cells). However, in clinical studies it has so far been mostly unsuccessful to evoke strong and durable T-cell responses through vaccination effective enough to eradicate e.g. an established tumour.

It was thus an object of the present invention to provide a novel method of evoking a more effective T-cell response through vaccination which preferably may be applied prophylactically or therapeutically to fight cancer or infectious diseases.

The object is solved by the embodiments described herein and as characterized by the claims.

The present invention provides improved vaccine compositions with improved immunogenicity. The present inventors have surprisingly found that by simultaneously co-vaccinating a peptide pair comprising of a short MHC I antigen (a first peptide) and of a long MHC II antigen (a second peptide) completely comprising the sequence of the first peptide (nested epitope), a significantly stronger and broader immune response is evoked in comparison to using only an MHC I or an MHC II antigen separately or to co-vaccinating unrelated MHC I and MHC II antigens.

According to the present invention, such MHC I and MHC II antigen pairs may be co-applied in form of peptides, nucleic acids (DNA or RNA) or vectors (viral, bacterial, or yeast/fungus) allowing their expression.

As demonstrated by the inventors using a set of 380 short MHC I and 262 long MHC II peptide epitopes which were derived from tumour-mutations and vaccinated into cancer patients (Example 1), co-vaccinating MHC I and MHC II epitope pairs according to the present invention leads to overall increase in the proportion of MHC II epitopes with T cell responses (see FIG. 1). Surprisingly when comparing T cell responses to all MHC II epitopes those which were co-vaccinated with the corresponding MHC I epitope showed higher proportion of CD4+ T cell responses (75% vs. 46%) and CD8+ T cell responses (19% vs. 11%) than MHC II epitopes vaccinated alone (FIG. 1.B).

The increased frequency of CD4+ T cell responses towards MHC II epitopes is surprising since the skilled person would expect that mainly CD8+ T cell responses towards MHC II epitopes should benefit from co-vaccination with the corresponding MHC I epitope which usually leads to CD8+ T cell activation. Accordingly, if there is a CD8+ T cell response to the MHC I epitope of a pair there should also be such a response to the MHC II epitope. However, the matched short MHC I epitopes which were co-vaccinated generally showed a slightly lower frequency of CD8+ T cell responses than MHC I epitopes vaccinated alone (9% vs. 12%; FIG. 2) and therefore this does not explain the increase in CD8+ T cell responses to the co-vaccinated MHC II epitopes. The latter may be partly explained by the fact that all MHC II epitopes which were co-vaccinated contain predicted MHC I epitopes, while this is only the case for a fraction of the MHC II epitopes vaccinated alone. Indeed, when comparing T cell responses of MHC II epitopes vaccinated alone those peptides which contain predicted short MHC I epitopes showed much higher frequency of CD8+ T cell responses than those lacking such MHC I epitopes (14% vs. 3%; FIG. 1.C). However, when comparing MHC II epitopes with predicted MHC I epitopes only, those which were co-vaccinated showed the highest frequency of CD8+ T cell responses (19% vs. 14%) although only 9% of their co-vaccinated MHC I epitopes efficiently induced such T cell responses, too. The only moderate increase in CD8+ T cell responses to co-vaccinated MHC II epitopes may hence be due to the fact that overall, the frequency of co-vaccinated MHC I epitopes with CD8+ T cell response was slightly lower than for the short epitopes vaccinated alone (9 vs. 12%; see FIG. 2).

That MHC I epitopes do not benefit more from co-vaccination of their cognate MHC II epitope (FIG. 2) is also considered surprising since it is well accepted that vaccinating MHC I epitopes together with an unrelated MHC II helper epitope may increase T cell responses towards the MHC I epitopes. Mechanistically CD4+ T cells activated by unrelated MHC II helper epitopes may secrete cytokines generally supporting the activation of nearby CD8+ T cells recognizing their MHC I epitope presented by APC.

The frequency of immunogenic short MHC I peptide epitopes may not increase with co-vaccination because the co-vaccinated predicted MHC I epitope contained in the long MHC II peptides may not be immunogenic. On the other hand, an MHC II peptide may contain several predicted MHC I epitopes including some which are and some which are not immunogenic. Therefore, selecting the right (immunogenic) nested MHC I epitope for co-vaccination would likely further lead to an increase in MHC I and MHC II epitopes with CD8+ T cell responses.

To further analyze the influence of predicted MHC I epitopes included in an MHC II epitope for a randomly selected, representative subset of the investigated MHC II epitopes (n=139) the number of in silico predicted nested MHC I epitopes was determined using the three epitope prediction algorithms NetMHC, NetMHCpan, and SYFPEITHI (Example 2). For MHC II epitopes which were not co-vaccinated with a corresponding MHC I epitope increasing the number of included predicted MHC I epitopes to >=5, also increased markedly the number of mainly CD4+ T cell responses to these MHC II epitopes (from 35% to 62%) while CD8+ T cell responses remained similar (22% vs 24%; FIG. 10). This is unexpected since with increasing number of nested MHC I epitopes included in an MHC II epitope also the likelihood increases that an immunogenic MHC I epitope eliciting a CD8+ T cell response is among them. Surprisingly, frequencies of both CD8+ and CD4+ T cell responses towards MHC II epitopes could be further and largely improved when these included >=5 predicted MHC I epitopes and were co-vaccinated with one of these MHC I epitopes. In total 74% of such co-vaccinated MHC II epitopes showed CD4+ T cell responses and 41% CD8+ T cell responses (FIG. 10). As only 12% of the matched co-vaccinated MHC I epitopes elicited a CD8+ T cell response (FIG. 8), this does not explain the increase of such T cell responses to the co-vaccinated long epitopes.

Also the strength of CD8+ T cell responses are significantly increased for immunogenic MHC I epitopes which were co-vaccinated with the corresponding MHC II epitope compare to immunogenic MHC I epitopes vaccinated alone (FIG. 11). Finally, there was also a trend for slightly stronger CD4+ T cell responses to immunogenic MHC II epitopes when co-vaccinated with the corresponding MHC I epitope compared to immunogenic MHC II epitopes vaccinated alone (FIG. 12). This trend of slightly stronger CD4+ T cell activation after co-vaccination was seen in both groups of MHC II epitopes namely those containing 1-4 or more than 4 predicted MHC I epitopes.

In summary co-vaccinating a peptide pair consisting of an MHC I epitope and an MHC II epitope, where the MHC II epitope completely includes the sequence of the MHC I epitope, leads to surprisingly high rates of CD4+ and CD8+ T cell responses to the MHC II epitopes. This rate may be further increased if MHC II epitopes are selected which contain several predicted MHC I epitopes and co-vaccinated with at least one of said predicted MHC I epitopes. Co-vaccination also increased the strength of T cell responses to both, MHC I and MHC II epitopes of a pair.

Generally, it is considered that immunizations with vaccines are most efficient, if both strong CD4+ and CD8+ T cell responses are induced since both immune cell populations have complementary functions and also may support each other's actions leading to the most profound immune response against a pathogen or tumour. The present invention describes now a novel combination of co-vaccinated MHC I and MHC II epitope pairs which have the propensity to increase strength and frequency of vaccine-induced CD4+ and CD8+ T cell responses and may therefore be beneficially applied for prevention or treatment of an infectious disease or cancer.

The present invention will be summarized in the following embodiments.

In a first embodiment, the present invention relates to a vaccine composition comprising a first peptide or a nucleic acid encoding the first peptide that can be expressed upon administration of said nucleic acid to a subject, wherein the first peptide is an MHC I antigen; and a second peptide or a nucleic acid encoding the second peptide that can be expressed upon administration of said nucleic acid to a subject, wherein the second peptide is an MHC II antigen; wherein the sequence of the first peptide is comprised in the sequence of the second peptide.

In a second embodiment, the present invention relates to a kit of parts comprising a vaccine composition comprising a first peptide or a nucleic acid encoding the first peptide that can be expressed upon administration of said nucleic acid to a subject, wherein the first peptide is an MHC I antigen; and a vaccine composition comprising a second peptide or a nucleic acid encoding the second peptide that can be expressed upon administration of said nucleic acid to a subject, wherein the second peptide is an MHC II antigen; wherein the sequence of the first peptide is comprised in the sequence of the second peptide.

In a third embodiment, the present invention relates to the vaccine composition of the present invention or the kit of parts of the present invention for use as a vaccine. Said vaccine may be a prophylactic vaccine applied before the onset of a disease or a therapeutic vaccine applied after the onset of a disease.

In a fourth embodiment, the present invention relates to the vaccine composition of the present invention or the kit of parts of the present invention for use in a method of inducing an immune response in a subject.

In a fifth embodiment, the present invention relates to the vaccine composition of the present invention or the kit of parts of the present invention for use in a method of increasing an immune response in a subject.

In a sixth embodiment, the present invention relates to the vaccine composition of the present invention or the kit of parts of the present invention for use in therapy. The therapy may refer to the treatment/therapy of an infectious disease.

In a seventh embodiment, the present invention relates to the vaccine composition of the present invention or the kit of parts of the present invention for use in cancer therapy.

In an eighth embodiment, the present invention relates to a method for preparing a subject-specific vaccine composition for use in cancer therapy, the method comprising the steps of
(a) obtaining tumour and normal DNA or tumour and normal RNA sequence information for a subject diagnosed with cancer;
(b) identifying subject-specific tumour-mutations and subject-specific MHC I and MHC II restricted tumour neoantigens based on the sequence information obtained in (a);
(c) determining the sequence of a first peptide, wherein the first peptide is an MHC I antigen and its sequence comprises a sequence comprising a subject-specific tumour neoantigen identified in step (b);
(d) determining the sequence of a second peptide, wherein the second peptide is an MHC II antigen, its sequence comprises a sequence comprising a subject-specific tumour neoantigen identified in step (b) and comprises the sequence of the first peptide;
(e) preparing the first peptide or a nucleic acid encoding the first peptide that can be expressed upon administration of said nucleic acid to a subject;
(f) preparing the second peptide or a nucleic acid encoding the second peptide that can be expressed upon administration of said nucleic acid to a subject; and
(g) formulating the subject-specific vaccine composition using the preparations of (e) and (f).

In a ninth embodiment the present invention relates to a method of inducing an immune response in a subject, the method comprising administering the vaccine composition of the present invention or the parts of the kit of parts of the present invention to a subject in need thereof.

In a tenth embodiment, the present invention relates to a method of increasing an immune response in a subject, the method comprising administering the vaccine composition of the present invention or the parts of the kit of parts of the present invention to a subject in need thereof.

In an eleventh embodiment, the present invention relates to a method of treating a cancer disease in a subject, the method comprising administering the vaccine composition of the present invention or the parts of the kit of parts of the present invention to a subject in need thereof.

In a twelfth embodiment, the present invention relates to use of a first peptide or a nucleic acid encoding the first peptide that can be expressed upon administration of said nucleic acid to a subject as provided in the present invention; and a second peptide or a nucleic acid encoding the second peptide that can be expressed upon administration of said nucleic acid to a subject as provided in the present invention; for a manufacture of a vaccine composition for use in a method of inducing an immune response in a subject.

In a thirteenth embodiment, the present invention relates to use of a first peptide or a nucleic acid encoding the first peptide that can be expressed upon administration of said nucleic acid to a subject as provided in the present invention; and a second peptide or a nucleic acid encoding the second peptide that can be expressed upon administration of said nucleic acid to a subject as provided in the present invention; for a manufacture of a vaccine composition for use in a method of increasing an immune response in a subject.

In a fourteenth embodiment, the present invention relates to use of a first peptide or a nucleic acid encoding the first peptide that can be expressed upon administration of said nucleic acid to a subject as provided in the present invention; and a second peptide or a nucleic acid encoding the second peptide that can be expressed upon administration of said nucleic acid to a subject as provided in the present invention; for a manufacture of a vaccine composition for use in a method of treating or preventing a cancer disease in a subject.

### BRIEF DESCRIPTION OF FIGURES

FIG. 1 illustrates the frequency of CD4+ and CD8+ T cell responses to 262 long MHC II epitopes derived from tumour-mutations and vaccinated into cancer patients together with other neoantigen peptides. MHC II peptide epitopes (also referred to herein as second peptide(s) or MHC II antigen(s)) were vaccinated alone or in combination with their cognate MHC I epitope as described in Example 1. For this analysis MHC II epitopes were discriminated according to the inclusion of any in silico predicted MHC I epitope (nested epitope) and co-vaccination status. The number of peptide epitopes per group is given in parentheses.

FIG. 2 illustrates the frequency of CD4+ and CD8+ T cell responses to 380 short MHC I epitopes derived from tumour-mutations and vaccinated into cancer patients together with other neoantigen peptides. MHC I peptide epitopes (also referred to herein as first peptide(s) or MHC I antigen(s)) were vaccinated alone or in combination with their cognate MHC II epitope as described in Example 1. The number of peptide epitopes per group is given in parentheses.

FIG. 3 presents a Table illustrating the use of a cancer vaccine wherein paired neoantigen peptides for 5 of 5 somatic mutations were co-vaccinated, i.e. administered to a subject as paired MHC I antigen and MHC II antigen derived from the same tumour mutation, as shown in Example 1.1. Several strong CD4+ and CD8+ T-cell responses were observed.

FIG. 4 presents a Table illustrating the use of a cancer vaccine wherein paired neoantigen peptides for 3 of 9 somatic mutations were co-vaccinated, i.e. administered to a subject as paired MHC I antigen and MHC II antigen derived from the same tumour mutation, as shown in Example 1.2. In this case two MHC I and one MHC II antigens were vaccinated for the PTEN mutant. For all other mutants one MHC I and one MHC II antigen were co-applied. Several strong CD4+ and CD8+ T-cell responses were observed.

FIG. 5 presents a Table illustrating the use of a cancer vaccine wherein paired neoantigen peptides for 1 of 9 somatic mutations were co-vaccinated, i.e. administered to a subject as paired MHC I antigen and MHC II antigen derived from the same tumour mutation, as shown in Example 1.3. A moderate CD4+ response was observed against an MHC II antigen vaccinated as pair and no CD8+ T responses were observed.

FIG. 6 presents a Table illustrating the use of a cancer vaccine wherein paired neoantigen peptides for 1 of 9 somatic mutations were co-vaccinated, i.e. administered to a subject as paired MHC I antigen and MHC II antigen derived from the same tumour mutation, as shown in Example 1.4. A single CD4+ T-cell response was observed against an MHC II antigen vaccinated as pair, but no CD8+ T-cell responses were observed.

FIG. 7 presents a Table illustrating a use of a cancer vaccine wherein for none of 10 targeted somatic mutations paired neoantigen peptides were co-vaccinated, i.e. administered to a subject as paired MHC I antigen and MHC II antigen derived from the same tumour mutation, as shown in Example 1.5. No immune responses were observed.

FIG. 8 shows the frequency of CD4+ and CD8+ T cell responses to 317 short MHC I epitopes derived from tumour-mutations and vaccinated into cancer patients together with other neoantigen peptides. For this analysis MHC I epitopes were discriminated according to the co-vaccination status as described in Example 2. The number of peptide epitopes per group is given in parentheses.

FIG. 9 depicts the frequency of CD4+ and CD8+ T cell responses to 139 long MHC II epitopes derived from tumour-mutations and vaccinated into cancer patients together with other neoantigen peptides. For this analysis MHC II epitopes were discriminated according to the inclusion of any in silico predicted MHC I epitope (nested epitope) and co-vaccination status as described in Example 2. The number of peptide epitopes per group is given in parentheses.

FIG. 10 shows the frequency of CD4+ and CD8+ T cell responses to 139 long MHC II epitopes derived from tumour-mutations and vaccinated into cancer patients together with other neoantigen peptides. For this analysis MHC II epitopes were discriminated according to the number of included in silico predicted MHC I epitopes (nested epitopes) and co-vaccination status as described in Example 2. The number of peptide epitopes per group is given in parentheses.

FIG. 11 shows the strengths of CD8+ and CD4+ T cell responses to short MHC I epitopes discriminated according to the co-vaccination status as described in Example 2. Shown are % activated CD8+ or CD4+ T cells after incubation of PBMC with the respective short MHC I peptide epitope, intracellular cytokine staining, and FACS analysis. Included were only epitopes with an immune response. Provided p-values are from Mann-Whitney testing without multiple test correction. The number of peptide epitopes per group is given in parentheses.

FIG. 12 shows the strength of CD4+ T cell responses to long MHC II epitopes discriminated according to the number of included in silico predicted MHC I epitopes (nested epitopes) and co-vaccination status as described in Example 2. Shown are % activated CD4+ T cells after incubation of PBMC with the respective long MHC II peptide epitope, intracellular cytokine staining, and FACS analysis. Included were only epitopes with an immune response. The number of peptide epitopes per group is given in parentheses.

### DETAILED DESCRIPTION OF THE INVENTION

The compositions, kits of parts, uses and methods of the invention will be described in the following. It is to be understood that all the combinations of features are envisaged.

In a first embodiment, the present invention relates to a vaccine composition. The vaccine composition of the present invention comprises a first peptide or a nucleic acid encoding the first peptide that can be expressed upon administration of said nucleic acid to a subject, and a second peptide or a nucleic acid encoding the second peptide that can be expressed upon administration of said nucleic acid to a subject. The first peptide is an MHC I antigen, and the second peptide is an MHC II antigen. Furthermore, the sequence of the first peptide is comprised in the sequence of the second peptide.

The term “peptide” refers to a polymer of two or more amino acid residues linked via amide bonds that are formed between an amino group of one amino acid residue and a carboxylic acid group of another amino acid residue. The amino acid residues comprised in the peptide or protein, which are also referred to as amino acids, may be selected from the 20 standard proteinogenic α amino acids (i.e., Ala, Arg, Asn, Asp, Cys, Glu, Gln, Gly, His, Ile, Leu, Lys, Met, Phe, Pro, Ser, Thr, Trp, Tyr, and Val) but also from non-proteinogenic and/or non-standard α amino acids (such as, e.g., ornithine, citrulline, homolysine, pyrrolysine, 4 hydroxyproline, a methylalanine (i.e., 2 aminoisobutyric acid), norvaline, norleucine, terleucine (tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine) as well as β amino acids (e.g., β alanine), γ-amino acids (e.g., γ-aminobutyric acid, isoglutamine, or statine) and δ-amino acids. Preferably, the amino acid residues comprised in the peptide or protein are selected from α amino acids, more preferably from the 20 standard proteinogenic α amino acids (which can be present as the L isomer or the D-isomer, and are preferably all present as the L isomer). The peptide may be unmodified or may be modified, e.g., at its N terminus, at its C terminus and/or at a functional group in the side chain of any of its amino acid residues (particularly at the side chain functional group of one or more Lys, His, Ser, Thr, Tyr, Cys, Asp, Glu, and/or Arg residues). Such modifications may include, e.g., the attachment of any of the protecting groups described for the corresponding functional groups in: Wuts P G & Greene T W, Greene's protective groups in organic synthesis, John Wiley & Sons, 2006. Such modifications may also include the covalent attachment of one or more polyethylene glycol (PEG) chains (forming a PEGylated peptide), the glycosylation and/or the acylation with one or more fatty acids (e.g., one or more C8-30 alkanoic or alkenoic acids; forming a fatty acid acylated peptide or protein). Moreover, such modified peptides or proteins may also include peptidomimetics, provided that they contain at least two amino acids that are linked via an amide bond (formed between an amino group of one amino acid and a carboxyl group of another amino acid). The amino acid residues comprised in the peptide or protein may, e.g., be present as a linear molecular chain (forming a linear peptide) or may form one or more rings (corresponding to a cyclic peptide). Unless indicated to the contrary, the peptides as referred to herein are linear peptides of less than 40 residues, comprising amino acid residues selected from the 20 standard proteinogenic α amino acids, each present as L isomer. Preferably, side chains of amino acid residues are not modified. Preferably, N-terminus of the peptide is either not modified or is acetylated, and/or C-terminus of the peptide is either not modified or is amidated. Acetylation is preferably understood herein as a modification of the amino group, preferably a main chain amino group NH₂—, into CH₃CONH— moiety. Amidation is preferably understood herein as a modification of the carboxylic group, preferably a main chain carboxylic group —COOH, into a —CONH₂ moiety.

As understood herein, the sequence of the peptide is a one-dimensional representation of the amino acid residues comprised in the polypeptide chain, indicating the order of different types of residues, preferably from the N-terminus to the C-terminus of said peptide. The skilled person understands different conventions in describing the amino acid sequence of a peptide, for example a Fasta sequence format utilizing a single letter code. It is to be understood that preferably when reference is made to a sequence of a peptide, non-amino acid residue modifications of N-terminus and/or C-terminus of the peptide are not to be taken into account. Thus, in other words, the sequence of a peptide is understood as referring to the amino acid composition of said peptide only.

As understood herein, peptides components, i.e., the first peptide and/or the second peptide, may be present in a vaccine formulation as purified peptides. As understood to the skilled person, peptides are obtainable according to the art of chemical peptide synthesis. Peptides are typically obtainable according to the methods of solution peptide synthesis or solid phase peptide synthesis. To date, solid phase peptide synthesis has become standard practice for chemical peptide synthesis.

Solid phase peptide synthesis is a process used to chemically synthesize peptides on solid supports. In solid phase peptide synthesis, an amino acid or peptide is bound, usually via the C-terminus, to a solid support. New amino acid residues are added to the bound amino acid residue or peptide via coupling reactions. Due to the possibility of unintended reactions, protection groups are typically used. The broad utility of solid phase peptide synthesis has been demonstrated by the commercial success of automated solid phase peptide synthesizers. As understood to the skilled person, a peptide of substantially any sequence, preferably up to 50 amino acid residues, can be readily obtained by using the means of solid phase peptide synthesis by using the routine experimentation.

As known to the skilled person, when formulating a vaccine composition, not only peptides, but also nucleic acids (e.g. DNA, RNA) or vectors (e.g. viral, bacterial, yeast/fungal), as well as recombinant proteins can be used for immunization. However, T-cells always ultimately recognize MHC-presented peptides, said peptides originating from proteolytic cleavage of larger peptides, recombinant proteins or proteins/peptides encoded and expressed by a nucleic acid or vector.

Thus, in the vaccine composition of the present invention, the first peptide is preferably present in a form of a peptide or a pharmaceutically acceptable salt thereof.

Further accordingly, in the vaccine composition of the present invention, the second peptide is preferably present in a form of a peptide or a pharmaceutically acceptable salt thereof.

As understood herein, pharmaceutically acceptable salt forms of the peptides of the present invention may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2 naphthalenesulfonate (napsylate), 3 phenylsulfonate, or camphorsulfonate salts; glycerophosphate salts; and acidic amino acid salts such as aspartate or glutamate salts. Preferred pharmaceutically acceptable salts of the peptides of the present invention include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the peptides as described herein is a hydrochloride or an acetate salt.

As understood herein, the peptides may also be applied in a form of a nucleic acid encoding the same, which can be expressed (as understood herein, expressed in a subject), upon administration of said nucleic acid to a subject. Examples of nucleic acids suitable for use in the present invention include an mRNA encoding an antigen, or a DNA vector encoding said antigen. Preferentially paired MHC I and MHC II antigens derived from the same tumour mutation, unmutated tumour antigen or pathogen protein are encoded on the same nucleic in order to ensure combined application, uptake, expression and presentation by the same APC.

An mRNA vaccine (which may also be referred to as RNA vaccine) uses a molecule of messenger RNA to produce an immune response. A short lived, preferably synthetically created mRNA molecule encoding an antigen (herein it would be the first peptide or the second peptide or both) is taken up by APCs and use the cellular ribosomes to produce the peptide antigen encoded by said mRNA (or a protein comprising said antigen, whereas the peptides antigen will be produced by e.g. cellular proteasomes afterwards). Preferably, mRNA for a vaccine use is produced by using the RNA synthesis methods known to the skilled person, for example solid state RNA synthesis or in vitro transcription. It will be understood that said RNA molecule may include nucleoside modifications, aimed at e.g. improving the stability of the mRNA molecule. The so modified RNA molecules are considered also to be included.

Thus, in one specific embodiment, the first peptide is present in a form of a nucleic acid encoding the first peptide that can be expressed upon administration of said nucleic acid to a subject.

In a further specific embodiment, the second peptide is present in a form of a nucleic acid encoding the second peptide that can be expressed upon administration of said nucleic acid to a subject.

Thus accordingly, preferably the present invention relates to a vaccine composition, comprising a first peptide, wherein the first peptide is an MHC I antigen; and a second peptide, wherein the second peptide is an MHC II antigen; wherein the sequence of the first peptide is comprised in the sequence of the second peptide.

The present inventors have found that, surprisingly, administration of the first peptide together with the second peptide is particularly preferred. Thus, in an embodiment of the present invention wherein the first peptide is present in a form of a nucleic acid encoding the first peptide that can be expressed upon administration of said nucleic acid to a subject and wherein the second peptide is present in a form of a nucleic acid encoding the second peptide that can be expressed upon administration of said nucleic acid to a subject, the nucleic acid encoding the first peptide and the nucleic acid encoding the second peptide are preferably so formulated to be administered together.

Thus, in one specific embodiment, the first peptide and the second peptide are present in a form of a single nucleic acid that encodes both the first peptide and the second peptide, and thus is configured to express both the first peptide and the second peptide together or as separate entities upon administration of said nucleic acid to a subject.

Within the scope of the present invention, in the vaccine composition of the present invention, the first peptide is an MHC I antigen and the second peptide is an MHC II antigen.

In the adaptive immune response, foreign antigens are recognized by receptor molecules on B lymphocytes (e.g., immunoglobulins) and T lymphocytes (e.g., T-cell receptor or TCR). These foreign antigens are presented on the surface of cells as peptides by specialized complexes, generically referred to as major histocompatibility complex (MHC) molecules. MHC molecules are encoded by multiple loci that are found as a linked cluster of genes that spans about 4 Mb. In humans, the genes are found on chromosome 6 and are called human leukocyte antigen (HLA) genes. The loci in humans are polygenic and include three highly polymorphic classes of MHC genes (class I, II and III).

MHC II molecules are exclusively expressed on antigen-presenting cells (APC) such as B cells, monocytes, macrophages and dendritic cells which can phagocytize exogenous material, while MHC I molecules are expressed more ubiquitously in virtually all somatic cells. MHC I displays endogenous antigens (like unmutated or mutated tumour antigens), or antigens from within the cell (e.g. if a virus replicates inside a cell), while MHC II displays exogenous antigens, or antigens from outside the cell which were taken up by APC via phagocytosis. However, APCs have also the ability to shuttle exogenous antigens not only to the MHC II presentation pathway but also for MHC I presentation. This process is also called cross-presentation.

Since MHC I molecules are expressed on all nucleated cells, they are also expressed in tumour cells. MHC I molecules display protein fragments (typically peptides of 8 to 13 amino acid residues long, preferably 8 to 10 amino acids residues long) on the surface to CD8+ cytotoxic T lymphocytes (CTLs). CTLs are specialized to kill any cell that presents an MHC I-bound peptide recognized by its own membrane-bound T-cell receptor (also referred to as TCR). T-cells with TCRs recognizing peptides from “normal” cellular proteins (unmutated and not from pathogen origin) are usually removed during their maturation in the thymus by negative selection, thereby generating a repertoire of peripheral T-cells that is largely self-tolerant. When a cell displays via MHC I peptides derived from proteins that are not normally present (e.g., of viral, tumour, or other non-self origin), such cells may be recognized and killed by a CTL. Vaccination with MHC I peptides has the goal to activate and expand CTL (CD8+ T cells) having the capacity to recognize and kill cells presenting their target peptide via MHC I.

MHC I molecules are known to the skilled person to have a closed peptide binding cleft which can harbour peptides of the length of about 9-10 amino acid residues. The closed conformation limits the size of peptides which can be bound. Usually, the second and the last amino acid of an antigenic peptide make direct contact to the MHC I molecule and are responsible for the binding. Hence peptides shorter than 8 amino acid residues in length would struggle to anchor within the antigen-binding cleft. It is further known to the skilled person that, generally, the MHC genes show high genetic variability and this leads to different peptide binding preferences. Prediction algorithms have been developed which were trained on in vitro binding data of MHC I molecules and peptides. These algorithms are capable of predicting whether a peptide may bind to a certain MHC I molecule. More recently, further insight about length distribution of MHC I-restricted peptides came from ligandome analyses. Here the peptide/MHC complexes from cell cultures or tissue samples are precipitated and the eluted peptides are identified by mass spectrometry. Such data convincingly show that MHC I bound peptides have a length between 8 and 13 amino acid residues with a peak at 9 amino acid residues.

An MHC I antigen is thus a peptide that can bind to MHC I receptor and be presented to CD8+ T cells (CTLs). Thus, said MHC I antigen may also be referred to as an CD8 antigen. Preferably, an MHC I antigen is 8 to 13 amino acid residues long. More preferably, an MHC I antigen is 8 to 10 amino acid residues long. It is noted that antigens (peptides) displayed by MHC I receptor are usually peptides of cytosolic origin, i.e. originate from inside the cell, but may also be presented by MHC I molecules when delivered exogenously, e.g. by vaccination of a short MHC I peptide or a long peptide or a protein containing a short MHC I binding peptide, where the long peptides or protein need to be taken up by the cell and processed to obtain the comprised short MHC I peptide before MHC I-mediated presentation.

MHC II, also referred to as class II MHC molecules, are a class of major histocompatibility complex (MHC) molecules normally found only on professional antigen-presenting cells (APC) such as B cells, monocytes, macrophages and dendritic cells. These cells are important in initiating immune responses. The antigens presented by APC via MHC II peptides are usually derived from extracellular proteins (not from cytosolic proteins as in case of MHC class I molecules), like from extra-cellular pathogens, cell debris from dead pathogen infected or cancer cells or from vaccinated long peptides or proteins containing a long MHC II peptide, where the long peptides or proteins may need to be taken up by the APC and processed to obtain the comprised long MHC II peptide before MHC II-mediated presentation.

In humans, the MHC class II protein complex is encoded by the human leukocyte antigen gene complex (HLA). HLAs corresponding to MHC class II are HLA-DP, HLA-DQ, and HLA-DR. MHC II peptide complexes are recognized by T helper cells (CD4+).

Although the peptide binding cleft of MHC II molecules is of similar size like for the MHC I molecules (harbouring peptides of 9-10 amino acid residues), peptide length preferences for MHC II binding are largely different. This is mainly due to the fact that the MHC class II antigen-binding cleft is open at both ends, allowing antigens to protrude from both ends of the antigen-binding cleft. Ligandome data show that the most abundant MHC II bound peptides are of size 15 amino acid residues ranging mainly between 14 and 35 amino acid residues.

The peptide presented by the MHC II complex, is also referred to as MHC II antigen, or CD4 antigen. Preferably, an MHC II antigen is at least 14 amino acid residues long. More preferably, an MHC II antigen is 14 to 35 amino acid residues long. Even more preferably, an MHC II antigen is 14 to 25 amino acid residues long.

In summary, by definition MHC I and MHC II epitopes are restricted to the binding of either MHC I or MHC II molecules, respectively. Binding specificity is defined by the peptide size and the interaction of specific amino acid residues of the peptide with the peptide binding pocket of the MHC molecule.

Thus preferably, as referred to herein, the MHC I antigen is preferably to be understood as MHC I-restricted antigen. Accordingly, the MHC-II antigen is preferably to be understood as MHC II-restricted antigen. It is to be understood that an MHC I-restricted antigen does not bind to, or substantially does not bind to, or detectably does not bind to, MHC II, and that MHC II-restricted antigen does not bind to, or substantially does not bind to, or detectably does not bind to, MHC I. It is however noted that a long MHC II antigen peptide may be cleaved inside the cell resulting in one or more shorter peptides, which could bind to the MHC I molecules of that person. Accordingly, upon such cleavage, an MHC II antigen peptide may elicit a response characteristic to an MHC I antigen (i.e. a CD8+ T cell response).

Thus, within the scope of the present invention, in the vaccine composition of the present invention the first peptide is an MHC I antigen (also referred to as CD8 antigen). In other words, the first peptide may be presented on the MHC I complex. Preferably, the first peptide is 8 to 13 amino acid residues long. More preferably, the first peptide is 8 to 10 amino acid long.

Further accordingly, within the scope of the present invention, the second peptide is an MHC II antigen (also referred to as CD4 antigen). In other words, the second peptide may be presented on the MHC II complex. Preferably, the second peptide is at least 14 amino acid residues long. More preferably, the second peptide is 14 to 35 amino acid residues long. Even more preferably, the second peptide is 14 to 25 amino acid residues long.

According to the present invention, in particular in the vaccine composition of the present invention, the sequence of the first peptide is comprised in the sequence of the second peptide. In other words, it is possible to select a sub-sequence within the sequence of the second peptide that is identical with the sequence of the first peptide, or with a fragment thereof that is at least 4, at least 5, at least 6, at least 7, at least 8, at least 9, at least 10, at least 11, at least 12, or at least 13 amino acid residues long, preferably it is possible to select a sub-sequence within the sequence of the second peptide that is identical with the sequence of the first peptide. According to the present invention, the sequence of the first peptide (or the fragment thereof) may be comprised in the sequence of the second peptide in any way. Thus, the sequence of the second peptide may be obtainable from the sequence of the first peptide upon addition of amino acid residue(s) to both its N-terminus and C-terminus. However, alternatively, the sequence of the second peptide may also be obtainable from the sequence of the first peptide upon addition of the amino acid residues to either its N-terminus or its C-terminus.

Preferably, the sequence of the first peptide being comprised in the sequence of the second peptide may in other words be described as that the first peptide as described in the present invention is nested in the second peptide as described in the present invention. The present invention may further relate to an embodiment wherein the composition comprises more than one MHC I peptide that fulfils the definition of the first peptide, as referred to herein, and a second MHC II peptide, wherein the sequence of each of the “first peptides” is comprised in the sequence of the second peptide. Accordingly, and preferably, the composition of the invention preferably comprises more than one first peptide, wherein the sequence of each of the first peptides is comprised in the sequence of the second peptide. In this situation, one may refer to a second peptide, comprising more than one nested MHC I epitope, wherein at least one of any of these nested MHC I epitopes, or a combination thereof are co-vaccinated (in a form of separate “first peptides”) together with said second peptide. The second peptide according to the invention may comprise 2, 3, 4, 5 or more than 5 nested MHC I epitopes.

The skilled person with an objective of determining whether a particular sequence is or can be an MHC I antigen or MHC II antigen would employ available bioinformatic prediction algorithms, which are capable making said determination. Such algorithms usually predict binding or presentation of antigens by the MHC molecules. Such bioinformatic prediction algorithms that can be used herein include e.g., but not exclusively for MHC I binding and/or presentation prediction NetMHC, NetMHCpan, MHCflurry, Puffin, SYFPEITHI, and/or MixMHCpred and for MHC II binding and/or presentation prediction NetMHCII, NetMHCIIpan, MixMHC2pred, MARIA, SYFPEITHI, TEPITOPE, SMM-Align, BERTMHC and/or Multipred.

Thus preferably, in the vaccine composition of the present invention, the first peptide is determined to be an MHC I antigen by using a bioinformatic prediction algorithm (i.e., one or more bioinformatic epitope prediction algorithm). Such bioinformatic MHC I epitope prediction algorithms are preferably selected from NetMHC, NetMHCpan, SYFPEITHI, Puffin, MixMHCpred and MHCflurry.

Further preferably, in the vaccine composition of the present invention, the second peptide is determined to be an MHC II antigen by using a bioinformatic prediction algorithm (i.e., one or more bioinformatic epitope prediction algorithm). Such bioinformatic MHC II epitope prediction algorithms are preferably selected from NetMHCII, NetMHCIIpan, MixMHC2pred, MARIA, SYFPEITHI, TEPITOPE, SMM-Align, BERTMHC and Multipred.

As encompassed by the present invention, the first peptide is determined to be an MHC I antigen by using a bioinformatic prediction algorithm, as described hereinabove, and the second peptide is selected as a peptide whose sequence comprises the sequence of the first peptide and the sequence of the second peptide is at least 14 amino acid residues long, preferably is 14 to 35 amino acid residues long, and preferably has been determined by MHC II antigen prediction algorithms.

Accordingly, the skilled person when practicing the present invention is capable of selecting the first and the second peptide of the present invention that fulfil the following requirements: the first peptide is an MHC I antigen (preferably MHC I-restricted antigen, as provided herein, preferably determined so by using a bioinformatic prediction algorithm, as provided herein); the second peptide is an MHC II antigen (preferably MHC II-restricted antigen, as provided herein, preferably determined so by using a bioinformatic prediction algorithm, as provided herein), and the sequence of the first peptide is comprised in the sequence of the second peptide. The skilled person appreciates that each peptide may also be present in a form of a nucleic acid encoding said peptide separately or both peptides together that can be expressed upon administration of said nucleic acid to a subject.

Accordingly and preferably, as provided in the present invention, the first peptide is configured to be an MHC I antigen (preferably an MHC I-restricted antigen), preferably determined so by using a bioinformatic prediction algorithm, as provided herein; the second peptide is configured to be an MHC II antigen (preferably an MHC II-restricted antigen), preferably determined so by using a bioinformatic prediction algorithm, as provided herein, and the sequence of the first peptide is comprised in the sequence of the second peptide.

Preferably, in the case of the second peptide comprising more than one nested MHC I epitope, the prediction whether or not a subsequence in the sequence of said second peptide is an MHC I epitope can be done using a bioinformatic prediction algorithm, as provided herein, for each continuous subsequence that can be selected from the sequence of the second peptide.

Particularly advantageous is a vaccine composition comprising a second peptide (or a nucleic acid encoding the second peptide that can be expressed upon administration of said nucleic acid to a subject), wherein the second peptide is an MHC II antigen and wherein the sequence of the second peptide comprises more than one sequence of an MHC I antigen, and a first peptide (or a nucleic acid encoding the first peptide that can be expressed upon administration of said nucleic acid to a subject), wherein the first peptide is an MHC I antigen with a sequence selected from sequences of MHC I antigens comprised in the sequence of the second peptide.

The first peptide and the second peptide are as defined hereinabove.

Preferably, the second peptide comprises two, three, four, five or more than five sequences of an MHC I antigen. It is to be understood that said sequences, each comprised in the sequence of the second peptide, may overlap. Accordingly, in the second peptide comprising more than one sequence of an MHC I antigen, for example two, three, four, five or more than five sequences of an MHC I antigen, the skilled person (optionally using bioinformatics prediction algorithms, as defined herein) may select a sequence of an MHC I antigen in more than one way, for example in two, three, four, five or more than five ways. Each of the so selected sequences of an MHC I antigen may be used as a sequence of the first peptide, as provided in the present invention.

Particularly preferred is a second peptide comprising five or more than five sequences of an MHC I antigen, as provided herein.

Vaccine composition of the present invention may contain a pharmaceutically acceptable carrier, adjuvant, excipient and/or diluent.

Vaccine formulation may be formulated with one or more pharmaceutically acceptable excipients and/or carriers. The pharmaceutically acceptable excipient(s)/carrier(s) must be “acceptable” in the sense of being compatible with the other ingredients of the formulation and not deleterious to the recipient thereof. The formulations may conveniently be presented in unit dosage form and may be prepared by any method well known in the pharmaceutical art.

Suitable excipients used as carriers are typically large, slowly metabolised macromolecules such as proteins, saccharides, polylactic acids, polyglycolic acids, polymeric amino acids, amino acid copolymers, sucrose (Paoletti et al., 2001, Vaccine, 19:2118), trehalose (WO 00/56365), lactose and lipid aggregates (such as oil droplets or liposomes). Such carriers are well known to those of ordinary skill in the art. The vaccines may also contain excipients used as diluents, such as water, saline, glycerol, etc. Additionally, auxiliary substances, such as wetting or emulsifying agents, pH buffering substances, and the like, may be present. Sterile pyrogen-free, phosphate buffered physiologic saline is a typical carrier. Nontoxic organic solvents like dimethyl sulfoxide (DMSO) may be used to help dissolving drug substances like peptides. A thorough discussion of pharmaceutically acceptable excipients is available in reference Gennaro, 2000, Remington: The Science and Practice of Pharmacy, 20th edition, ISBN: 0683306472.

The vaccine composition of the present invention may further comprise additional adjuvants besides of the antigens or adjuvants may be applied independently at or near to the vaccine injection site. Vaccine preparation is generally described in Vaccine Design (“The subunit and adjuvant approach” (eds Powell M. F. & Newman M. J.) (1995) Plenum Press New York).

Suitable adjuvants include an aluminium salt such as aluminium hydroxide or aluminium phosphate, but may also be a salt of calcium, iron or zinc, or may be an insoluble suspension of acylated tyrosine, or acylated sugars, or may be cationically or anionically derivatised saccharides, polyphosphazenes, biodegradable microspheres, monophosphoryl lipid A (MPL), lipid A derivatives (e.g. of reduced toxicity), 3-O-deacylated MPL, quil A, Saponin, QS21, tocol (EP 0382271), Freund's Incomplete Adjuvant (Difco Laboratories, Detroit, MI), Montanide ISA 51 VG and Montanide ISA 720 VG (both from Seppic), Merck Adjuvant 65 (Merck and Company, Inc., Rahway, NJ), AS-2 (Smith-Kline Beecham, Philadelphia, PA), XS-15, poly-ICLC (OncoVir), CpG oligonucleotides, bioadhesives and mucoadhesives, microparticles, liposomes, polyoxyethylene ether formulations, polyoxyethylene ester formulations, muramyl peptides, or imidazoquinolone compounds (e.g. imiquimod and its homologues). Human immunomodulators suitable for use as adjuvants in the invention include cytokines such as interleukins (e.g. IL-1, IL-2, IL-4, IL-5, IL-6, IL-7, IL-12, etc), macrophage colony stimulating factor (M-CSF), tumour necrosis factor (TNF), granulocyte-macrophage colony-stimulating factor (GM-CSF, sargramostin) may also be used as adjuvants.

Preferably, sargramostim and/or imiquimod (Aldara) is used as an adjuvant in a vaccine composition or parts of a kit of parts of the present invention or applied independently at or near to the vaccination site.

Vaccine compositions of the invention may be lyophilised or in aqueous form, i.e. solutions or suspensions. Liquid formulations of this type allow the compositions to be administered direct from their packaged form, without the need for reconstitution in an aqueous medium, and are thus ideal for injection. Compositions may be presented in vials, or they may be presented in ready filled syringes. The syringes may be supplied with or without needles. A syringe will include a single dose of the composition, whereas a vial may include a single dose or multiple doses (e.g. 2 doses). As understood to the skilled person, the dose for an adult, adolescent, toddler, infant or less than one-year old human that may be administered by injection will be different.

The vaccine composition of the present invention may be an anti-tumour, antiviral, antifungal or antibacterial vaccine composition. As understood herein, the sequence of antigenic peptides, referred to herein as the first peptide and the second peptide, will determine the antigen against which the immune response is to be effected and thus will determine the purpose of the vaccine composition. In the case of an anti-tumour vaccine, the sequence of the first peptide and of the second peptide is preferably a sequence of a mutated or unmutated tumour antigen that preferably can be targeted by CD4+ and/or CD8+ T-cells. In the case of an anti-viral vaccine, the sequence of the first peptide and of the second peptide is preferably a sequence of a viral protein that preferably can be targeted by CD4+ and/or CD8+ T-cells. For example, proteins of the virus responsible for cell attachment and cellular entry are used to give raise to antigen sequences, but all other viral gene products may be similarly targeted. In the case of an anti-fungal vaccine, the sequence of the first peptide and of the second peptide is preferably a sequence of a fungal protein that preferably can be targeted by CD4+ and/or CD8+ T-cells. Accordingly, in the case of an anti-bacterial vaccine, the sequence of the first peptide and of the second peptide is preferably a sequence of a bacterial protein that preferably can be targeted by CD4+ and/or CD8+ T-cells. As known to the skilled person, certain anti-bacterial or anti-viral vaccines can be used to prevent a cancer disease in a subject.

The vaccine composition of the present invention may be a pathogen vaccine composition. The pathogen vaccine is a vaccine that is meant to activate the immune system of a subject to combat said pathogen. It is to be understood that in a pathogen vaccine composition of the present invention the sequence of the first peptide is derived from an antigen of a pathogen wherein the pathogen can be a virus, a bacterium or a fungus.

The vaccine composition of the present invention may be a cancer vaccine composition.

As understood herein, the cancer vaccine is a vaccine that is meant to activate the immune system of a subject suffering from a tumour, to act against said tumour. Treatment with a cancer vaccine may either treat existing cancer or prevent development of cancer. A vaccine that prevents development of cancer may be an anti-bacterial or anti-viral vaccine, as referred to hereinabove.

Typically, however, cancer vaccines are prepared by using and/or analysing the tumour sample obtained from a subject and are thus subject-specific to recognize and combat said tumour. Thus, within the scope of the present invention, a vaccine composition of the present invention is encompassed, wherein the sequence of the first peptide is derived from a mutated or unmutated tumour antigen. It is to be understood that the sequence of the second peptide is herein also derived from the same tumour antigen, and that the sequence of the second peptide comprises the sequence of the first peptide, as described herein.

Thus, in an embodiment of the present invention, the first peptide sequence is derived from a mutated or unmutated tumour antigen. It is to be understood that, accordingly, the sequence of the second peptide that comprises the sequence of the first peptide is also derived from the same tumour antigen as the sequence of the first peptide.

Preferably as understood herein, “derived sequence” means that a sequence of said peptide is identical to a fragment of sequence it is derived from.

Tumour antigens are known to the skilled person. Within the scope of the present invention, the tumour antigen may be an antigen that is aberrantly overexpressed in cancer tissue, e.g. Melan-A, NY-ESO-1, or NY-BR-1 antigens.

Melan-A, which is also known as melanoma antigen, is encoded in human by MLANA gene. A fragment of this protein, usually consisting of nine amino acid residues (positions 27 to 35 in the sequence of the protein), may be presented by MHC I complex on the surface of melanoma cells.

NY-ESO-1, which derives its name from New York esophageal squamous cell carcinoma-1, also referred to as cancer/testis antigen 1, is an example of cancer testis antigens. Further members of these family beyond NY-ESO-1 which may be relevant in cancer therapy include MAGE-A1, MAGE-A3, MAGE-A4, PRAME, CT83 and SSX2. Expression of these proteins is generally restricted to male germ cells in the adult subject. However, in cancer these developmental antigens are often re-expressed and can serve as a locus of immune activation.

NY-BR-1 is a differentiation antigen of the mammary gland that could be useful for diagnosis and/or immunotherapy of breast carcinomas. NY-BR-1 has been originally identified in a breast cancer patient.

In one embodiment of the vaccine composition of the present invention or the kit of parts of the present invention, the sequence of the first peptide is derived from an unmutated tumour-associated antigen.

Tumour antigen as discussed herein may also be a tumour mutation-derived neoantigen.

Thus preferably, within the scope of the present invention, the sequence of the first neoantigen peptide is derived from a protein harbouring one or more tumour-mutation(s) and comprises one or more of these tumour-mutation(s). It is to be understood that, accordingly, the sequence of the second neoantigen peptide that comprises the sequence of the first peptide is also derived from the same protein and is harbouring the identical tumour-mutation(s) as the sequence of the first neoantigen peptide.

As used herein the term “neoantigen” is an antigen that has at least one amino acid alteration that makes it distinct from the corresponding wild-type, parental antigen, e.g., via mutation in a tumour cell or additionally via an altered post-translational modification at or near the tumour mutation. A neoantigen can include a polypeptide sequence or a nucleotide sequence. A mutation can include a frameshift or in-frame (i.e., non-frameshift) insertion or deletion (Indel), missense substitution, splice site alteration, genomic rearrangement or gene fusion, or any genomic or expression alteration giving rise to a novel open reading frame (neoORF). A mutations can also introduce a splice variant. Post-translational modifications specific to a tumour cell can include aberrant phosphorylation and/or glycosylation. Post-translational modifications specific to a tumour cell can also include a proteasome-generated spliced antigen. See Liepe et al., a large fraction of HLA class I ligands are proteasome-generated spliced peptides; Science. 2016 Oct. 21; 354(6310):354-358.

As used herein the term “tumour neoantigen” is a neoantigen present in a subject's tumour cell or tissue but not in the subject's corresponding normal cells or tissues.

Thus preferably, in the vaccine composition of the present invention the sequence of the first peptide comprises a tumour-specific mutation. Preferably, said tumour-specific mutation is a missense mutation that e.g. leads to an altered amino acid sequence of expressed protein product. Further preferably, said cancer specific mutation is a subject specific and tumour-specific mutation. It is further preferred that said mutation is non-synonymous.

Thereby, the present invention provides a vaccine composition (in particular a cancer vaccine composition) that is tailored to a specific subject. In other words, the present invention provides a subject-specific vaccine composition, in particular a subject-specific cancer vaccine composition.

In a subject specific cancer vaccine composition of the present invention, preferably the sequence of the first peptide is a sequence derived from a subject-specific tumour mutation-derived neoantigen. Further preferably, it is to be understood that the cancer vaccine composition of the present invention is tailored to a specific subject by the sequence of the first peptide comprising a subject-specific and tumour-specific mutation.

In a further embodiment, the present invention relates to a kit of parts. The kit of parts of the present invention comprises a vaccine composition comprising a first peptide or a nucleic acid encoding the first peptide that can be expressed upon administration of said nucleic acid to a subject, and a vaccine composition comprising a second peptide or a nucleic acid encoding the second peptide that can be expressed upon administration of said nucleic acid to a subject. As encompassed by the invention, the first peptide as described herein is an MHC I antigen and the second peptide as described herein is an MHC II antigen. Furthermore, as understood herein, the sequence of the first peptide is comprised in the sequence of the second peptide.

As understood herein, peptide components, of each of the vaccine compositions forming the parts of the kit of parts, i.e., the first peptide and/or the second peptide, may be present in said vaccine formulations as purified peptides. The methods for obtaining said peptides are as described herein.

Thus, in the kit of parts of the present invention, the first peptide is preferably present in a form of a peptide or a pharmaceutically acceptable salt thereof.

Further accordingly, in the kit of parts of the present invention, the second peptide is preferably present in a form of a peptide or a pharmaceutically acceptable salt thereof.

Thus accordingly, the present preferably invention relates to a kit of parts comprising a vaccine composition comprising a first peptide, wherein the first peptide is an MHC I antigen; and a vaccine composition comprising a second peptide, wherein the second peptide is an MHC II antigen; wherein the sequence of the first peptide is comprised in the sequence of the second peptide.

As it however is also to be understood herein, in the kit of parts of the present invention the peptides may also be present in a form of a nucleic acid encoding the same, which can be expressed (as understood herein, expressed in a subject), upon administration of said nucleic acid to a subject. Examples of nucleic acids suitable for use in the present invention include an mRNA encoding one or both antigens (peptides), or a DNA vector encoding one or both antigens.

Thus, in one specific embodiment of the kit of parts of the present invention, the first peptide is present in a form of a nucleic acid encoding the first peptide that can be expressed upon administration of said nucleic acid to a subject.

In a further specific embodiment of the kit of parts of the present invention, the second peptide is present in a form of a nucleic acid encoding the second peptide that can be expressed upon administration of said nucleic acid to a subject.

In a further specific embodiment of the kit of parts of the present invention, the first peptide and the second peptide are both encoded by the same nucleic acid and both peptides can be expressed together upon administration of said nucleic acid to a subject.

Within the scope of the present invention, in the kit of parts of the present invention, the first peptide is an MHC I antigen and the second peptide is an MHC II antigen.

Thus, within the scope of the present invention, in the kit of parts of the present invention the first peptide is an MHC I antigen (also referred to as CD8 antigen). In other words, the first peptide may be presented on the MHC I complex. Preferably, the first peptide is 8 to 13 amino acid residues long. More preferably, the first peptide is 8 to 10 amino acid long.

Further accordingly, within the scope of the present invention, in the vaccine compositions of the parts of the kit of parts of the present invention the second peptide is an MHC II antigen (also referred to as CD4 antigen). In other words, the second peptide may be presented on the MHC II complex. Preferably, the second peptide is at least 14 amino acid residues long. More preferably, the second peptide is 14 to 35 amino acid residues long. Even more preferably, the second peptide is 14 to 25 amino acid residues long.

According to the present invention, in particular in the kit of parts of the present invention, the sequence of the first peptide is comprised in the sequence of the second peptide. In other words, it is possible to select a sub-sequence within the sequence of the second peptide that is identical with the sequence of the first peptide or with a fragment thereof that is at least 4, at least 5, at least 6, at least 7, at least 8, at least 9, at least 10, at least 11, or at least 13 amino acid residues long, preferably it is possible to select a sub-sequence within the sequence of the second peptide that is identical with the sequence of the first peptide. According to the present invention, the sequence of the first peptide may be comprised in the sequence of the second peptide in any way. Thus, the sequence of the second peptide may be obtainable from the sequence of the first peptide upon addition of amino acid residue(s) to both its N-terminus and C-terminus. However, alternatively, the sequence of the second peptide may also be obtainable from the sequence of the first peptide upon addition of the amino acid residues either to its N-terminus or to its C-terminus.

The skilled person with an object of determining whether a particular sequence is or can be an MHC I antigen or MHC II antigen would employ available bioinformatic prediction algorithms, which are capable making said determination, which is described hereinabove.

Thus preferably, in the kit of parts of the present invention, the first peptide is determined to be an MHC I antigen by using a bioinformatic prediction algorithm.

Further preferably, in the kit of parts of the present invention, the second peptide is determined to be an MHC II antigen by using a bioinformatic prediction algorithm.

As it is to be understood herein, vaccine compositions as encompassed in the kit of parts of the present invention may contain a pharmaceutically acceptable carrier, adjuvant, excipient and/or diluent. Said pharmaceutically acceptable carriers, adjuvants, excipients and/or diluents are described in detail hereinabove.

The kit of parts of the present invention may include vaccine compositions that are antiviral, antifungal, or antibacterial vaccine compositions. As understood herein, the sequence of antigenic peptides, referred to herein as the first peptide and the second peptide, will determine the antigen against which the immune response is to be effected upon administration of the said kit of parts, and thus will determine the purpose of the kit of parts comprising both vaccine compositions. In the case of the antiviral vaccine kit of parts comprising antiviral vaccine compositions, the sequence of the first peptide and of the second peptide is preferably a sequence of a viral protein that preferably can be targeted by CD4+ and/or CD8+ T-cells. For example, proteins of the virus responsible for cell attachment and cellular entry are used to give raise to antigen sequences (in other words, the sequence of the antigen is derived from the sequences of said virus protein that preferably can be targeted by CD4+ and/or CD8+ T cells). In the case of an antifungal vaccine, the sequence of the fungal protein that is available for targeting with the CD4+ and/or CD8+ T-cells is used to give raise to the antigen sequence. Accordingly, in the case of an antibacterial vaccine, the sequence of the bacterial protein that is available for targeting with the CD4+ and/or CD8+ T-cells is used to give raise to the antigen sequence. As known to the skilled person, certain antibacterial or antiviral vaccines can be used to prevent a cancer disease in a subject.

Thus, the kit of parts of the present invention may be a pathogen vaccine kit of parts. In said kit of parts, the vaccine compositions are pathogen vaccine compositions. The pathogen vaccine is a vaccine that is meant to activate the immune system of a subject to combat said pathogen. Accordingly, the first peptide sequence in one of parts of said kit of parts is derived from an antigen of a pathogen wherein the pathogen can be a virus, a bacterium, or a fungus.

The kit of parts of the present invention may be a cancer vaccine kit of parts, comprising cancer vaccine compositions.

Thus, in an embodiment of the kit of parts of the present invention, the first peptide sequence is derived from a mutated or unmutated tumour antigen. It is to be understood that, accordingly, the sequence of the second peptide that comprises the sequence of the first peptide is also derived from the same mutated or unmutated tumour antigen as the sequence of the first peptide.

Tumour antigen as discussed herein may also be a tumour-mutation derived neoantigen.

Thus preferably, in the kit of parts of the present invention, the sequence of the first neoantigen peptide is derived from a tumour-mutation. It is to be understood that, accordingly, the sequence of the second neoantigen peptide that comprises the sequence of the first peptide is also derived from the same tumour-mutation as the sequence of the first peptide.

Thus preferably, in the kit of parts of the present invention the sequence of the first peptide comprises a tumour-specific mutation. Preferably, said tumour-specific mutation is a missense mutation that e.g. leads to an altered amino acid sequence of expressed protein product. Further preferably, said tumour-specific mutation is a subject-specific tumour-specific mutation.

Thereby, the present invention provides a vaccine kit of parts comprising vaccine compositions (in particular a cancer vaccine kit of parts comprising cancer vaccine compositions) that is tailored to a specific subject. In other words, the present invention provides a subject-specific vaccine kit of parts vaccine comprising subject specific vaccine compositions, in particular a subject specific cancer vaccine kit of parts comprising cancer vaccine compositions.

In a subject specific cancer vaccine kit of parts of the present invention, preferably the sequence of the first peptide is a sequence derived from a subject-specific tumour neoantigen. Further preferably, it is to be understood that the cancer vaccine composition of the present invention is tailored to a specific subject by the sequence of the first peptide comprising a subject-specific and tumour-specific mutation.

The vaccine composition of the present invention as described hereinabove, as well as the kit of parts of the present invention as described hereinabove, can be used as a vaccine. The vaccine can be a prophylactic vaccine applied before the onset of a disease, or a therapeutic vaccine applied to treat said disease after manifestation. As preferably understood herein, a vaccine upon administration to a subject is meant to induce an immune response in said subject against a certain factor (or, in other words, entity; e.g. a virus, a microorganism, or a cancer present in the subject), preferably by exposing the immune system of said subject to an antigen comprised in said vaccine and/or delivered by administration of said vaccine. Said antigen (which is herein to be understood as at least one antigen, and may also refer to a plurality of antigens) is meant to be representative of said factor (entity) as referred to hereinabove, e.g. a virus, a microorganism or a cancer present in the subject and an immune response induced or increased against said antigen is equivalent to an immune response that could target said factor (entity).

As understood to the skilled person, inducing an immune response as referred to herein may also refer to increasing an immune response in a subject, in the case wherein said immune response was already present in said subject. In that case, a reference would be made to a booster vaccine.

Thus accordingly, the vaccine composition of the present invention as described hereinabove, as well as the kit of parts of the present invention as described hereinabove, can be used in a method of inducing an immune response in a subject. Further accordingly, the vaccine composition of the present invention as described hereinabove, as well as the kit of parts of the present invention as described hereinabove, can be used in a method of increasing an immune response in a subject.

As encompassed by the present invention, the immune response may be against any factor (entity). Thus, a vaccine composition is useful for prophylactic or therapeutic application against a tumour or an infectious disease, e.g. caused by a virus, or by a microorganism, preferably by a bacterium, a fungus or a protozoa, more preferably by a bacterium. In other words, a vaccine composition of the present invention is useful in the prevention or treatment of an infectious disease, e.g. caused by a virus, or by a microorganism, preferably by a bacterium, a protozoa or a fungus, more preferably by a bacterium.

The term “prevention” of a disorder or disease, as used herein, is well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g. genetic markers or phenotypic indicators. In case of cancer, this predisposition can be determined by sequencing of healthy tissues like blood in order to identify germline mutations predisposing the individual to develop cancer. In case of infectious diseases, the predisposition can be determined by factors including exposure to particular infectious agent, for example based on the environment of a subject, their working conditions, or being in contact with said agent or with persons infected by said agent. Furthermore, a factor that can be taken into account when discussing predisposition of a certain subject to a certain infectious disease is risk of long-term or acute health issues that may be caused by contracting particular infectious disease. In such case, one may refer to a subject being in particular risk upon potentially contracting a particular infectious disease.

It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a vaccine composition or the kit of parts of the present invention before any clinical and/or pathological symptoms are diagnosed or determined or can be diagnosed or determined by the attending physician.

As understood herein, inducing an immune response or increasing an immune response against a tumour or a particular pathogen, preferably against an infectious disease-causing pathogen, upon administration of the vaccine composition of the present invention or the kit of parts of the present invention, can also be referred to as preventing a disorder or disease, in particular a cancer disease and/or an infectious disease, preferably an infectious disease, in a subject that is administered with said vaccine composition of the invention or said kit of parts of the invention.

The term “treatment” of a disorder or disease, as used herein, is also well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief). In the case of treatment using the vaccine composition of the present invention or the kit of parts of the present invention, the treatment may also result, optionally in addition to any of the effects disclosed hereinabove, in preventing future occurrences of said disease or disorder, in particular of a cancer disease and/or an infectious disease.

The vaccine composition of the present invention or the kit of parts of the present invention comprising the vaccine compositions are to be administered to a subject according to the art. The dosage for an immunization generally occurs in a unit dosage range where the lower value is about 1, 5, 50, 500, or 1,000 μg of each peptide and the higher value is about 10,000; 20,000; 30,000; or 50,000 μg of each peptide. Dosage values for a human typically range from about 300 μg to about 50,000 μg of each peptide per 70 kilogram patient. Boosting dosages of between about 1.0 μg to about 50,000 μg of each peptide, administered pursuant to a boosting regimen over weeks to months, can be administered depending upon the patient's response and condition. Patient response can be determined for example by detecting neoantigen-specific CD4+ and/or CD8+ T-cells within the patient's peripheral blood.

In case the vaccine composition of the present invention or the corresponding kit of parts of the present invention comprise the first peptide and/or the second peptide in a form of a nucleic acid configured to express said peptide upon administration to a subject, the skilled person will be capable of adjusting the dosage of nucleic acid to maximize the antigen-specific immune response while minimizing unintended side effects and adverse events. Alternatively, the skilled person will be capable of adjusting the dosage of nucleic acid to a corresponding preferred dosage of peptide, as discussed hereinabove.

In case the vaccine composition of the present invention or the corresponding kit of parts of the present invention comprise the first peptide and/or the second peptide in a form of a vector (viral, bacterial or fungal) configured to express said peptide upon administration to a subject, the skilled person will be capable of adjusting the dosage of such vector to maximize the antigen-specific immune response while minimizing unintended side effects and adverse events. Alternatively, the skilled person will be capable of adjusting the dosage of such vector to a corresponding preferred dosage of peptide, as discussed hereinabove.

The vaccine compositions of the present invention as well as the parts of the kit of parts of the present invention can be administered to a subject according to any method known to the skilled person. Preferably, said vaccine compositions or said parts of the kit of parts are to be administered by intravenous injection, oral administration, intranasal administration, by inhalation, intraocular administration, by subcutaneous injection, by intradermal injection, by intramuscular injection or electroporation, by transdermal administration, by transmucosal administration, by intra-tumoural injection or by intranodal injection. Particularly preferred is administration by intradermal or subcutaneous injection. However, the herein recited modes of administration are not to be construed as limiting in any way.

The kit of parts of the present invention comprises two vaccine compositions. As it is to be understood herein, the vaccine compositions which together constitute a kit of parts of the present invention can be administered to a subject sequentially, separately or together. In a preferred embodiment both vaccine compositions of the kit of parts which are specific for the same antigen are administered together. In other words, preferably, the first peptide and the corresponding second peptide are administered together.

As understood herein sequential administration refers to administering the vaccine composition comprising the first peptide, followed by the administration of the vaccine composition comprising the second peptide. Alternatively, sequential administration may refer to administering the vaccine composition comprising the second peptide, followed by the administration of the vaccine composition comprising the first peptide. It is to be understood that both compositions can be so administered that one composition is administered immediately after another composition is administered or that there is a brief period of time separating both administrations, e.g. less than one hour, less than 30 minutes, less than 15 minutes or less than 5 minutes. Sequential administration may also mean that, in particular in the case of intramuscular administration, both compositions are preferably administered in the same muscle, preferably in the same place. Sequential administration may also mean that, in particular in the case of intradermal administration, both compositions are preferably administered in the same intra-cutaneous compartment of the skin, preferably in the same place. Sequential administration may also mean that, in particular in the case of subcutaneous administration, both compositions are preferably administered in the same subcutaneous compartment of the skin, preferably in the same place. As it is to be understood to the skilled person, sequential administration requires preferably administration of both vaccine compositions of the kit of parts during one session.

As understood herein, the administration together requires that the vaccine composition comprising the first peptide, and the vaccine composition comprising the second peptide, are mixed together before they are administered to the subject.

As further to be understood herein, the administration separately of both parts of the kit of parts requires that the vaccine composition comprising the first peptide and the vaccine composition comprising the second peptide are administered to the subject at a different time, preferably at different days, i.e. not during the same session. For example, one of the compositions can be administered 1, 2, 3, 4, 5, 6 or 7 days after the administration of another composition.

Preferably, the parts of the kit of parts of the present invention are to be administered together to a subject in need thereof.

The vaccine composition of the present invention as described hereinabove, as well as the kit of parts of the present invention as described hereinabove, can be used in therapy. In other words, the vaccine composition of the present invention as described hereinabove or the kit of parts of the present invention as described hereinabove can be used in the treatment of a disease or disorder. In particular, the compositions or the kits of the invention are particularly useful wherein the disease or disorder to be treated is cancer. Thus in particular, in one embodiment of the present invention the vaccine composition of the invention is a cancer vaccine composition, and the kit of parts of the invention is the cancer vaccine kit of parts.

Thus, the present invention further relates to the vaccine composition of the present invention or the kit of parts of the present invention for use in cancer therapy.

It is to be understood that the vaccine composition of the present invention or the kit of parts of the present invention can be used as a standalone therapeutic agent. However, the combination therapies including the vaccine composition of the present invention or the kit of parts of the present invention are also encompassed by the present invention. The vaccine composition of the present invention or the kit of parts of the present invention, in particular the cancer vaccine composition of the present invention or the cancer vaccine kit of parts of the present invention, can be administered in combination with but not limited to chemotherapy, immunotherapy, targeted therapy, hormone therapy, radiation therapy, and/or surgical treatment.

The vaccine composition or kit of parts comprising a vaccine composition of the present invention may be administered to a person suffering from a disease, in particular a cancer disease in order to treat such a disease.

In case of a cancer disease, the vaccine composition or kit of parts comprising a vaccine composition of the invention may be combined with therapies known to and applied by the skilled person in order to treat a subject suffering from a cancer disease, in order to improve clinical effectiveness of the vaccine and/or of the respective therapy administered before, in parallel or after the vaccination to said subject, which can be any one or any combination of the following therapies:
radiation therapy,
treatment with one or more further pharmaceuticals such as chemotherapy and/or anti-angiogenic drugs, preferably selected from Axitinib (Inlyta), Bevacizumab (Avastin), Cabozantinib (Cometriq), Everolimus (Afinitor), Lenalidomide (Revlimid), Lenvatinib mesylate (Lenvima), Pazopanib (Votrient), Ramucirumab (Cyramza), Regorafenib (Stivarga), Sorafenib (Nexavar), Sunitinib (Sutent), Thalidomide (Synovir, Thalomid), Vandetanib (Caprelsa) and/or Ziv-aflibercept (Zaltrap)) and/or targeted therapies like Afatinib (Gilotrif), Brigatinib (Alunbrig), Cetuximab (Erbitux), Cobimetinib (Cotellic), Dabrafenib (Tafinlar), Everolimus (Afinitor), Imatinib (Gleevec), Lapatinib (Tykerb), Olaparib (Lynparza), Osimertinib (Tagrisso), Palbociclib (Ibrance), Regorafenib (Stivarga), Rituximab (Rituxan, Mabthera), Rucaparib (Rubraca), Trametinib (Mekinist), Trastuzumab (Herceptin), and Vemurafenib (Zelboraf),
immunotherapies including administration of immune checkpoint inhibitors, e.g. targeting CTLA-4, PD-1, PD-L1 and/or targeting other immune checkpoints like CD27, CD28, CD40, CD137, GITR, ICOS, OX40, (or other stimulatory immune checkpoints), A2AR, CD272, CD276, IDO, KIR, VTCN1, LAG3, TIM-3, NOX2, VISTA (or other inhibitory immune checkpoints)) and/or oncolytic viruses like talimogene laherparepvec (T-VEC, Imlygic), pelareorep (Reolysin), HF10 (Canerpaturev-C-REV) and CVA21 (CAVATAK),
administration of immunostimulants activating innate or adaptive immunity like STING agonists (like ADU-S100, MK-1454, SB11285, MK-2118, E7766, DMXAA or ASA404), agonists for different Toll-like receptors (TLR) recognizing different pathogen-associated molecular patterns collectively called PAMPs (including TLR agonist like MALP-2, Pam2Cys2, XS-15, LPS, MPLA, poly-IC, poly-ICLC, Imiquimod, oligodeoxynucleotides (ODNs) containing CpG motifs), immune stimulatory cytokines (like GM-CSF, IL-2, IL-12, IL-15, IL-21, IFN-α), bacterial products like Live bacillus Calmette-Guerin (BCG), plant derived immunostimulants (like saponin, Montanide ISA 51 VG, and/or Montanide ISA 720 VG).

Preferably the vaccine composition or kit of parts comprising a vaccine composition of the invention may be combined with immune checkpoint inhibitors like pembrolizumab (Keytruda), nivolumab (Opdivo), cemiplimab (LIBTAYO), ipilimumab (Yervoy), atezolizumab (Tecentriq), avelumab (Bavencio), durvalumab (Imfinzi), tremelimumab and/or spartalizumab and/or relatlimab (BMS-986016).

The vaccine composition or kit of parts comprising a vaccine composition of the present invention may be administered to a person suffering from a disease, in particular an infectious disease (e.g. caused by a viral, bacterial or fungal infection) in order to treat such a disease.

As it is to be understood herein, an infectious disease is preferably a viral, bacterial or fungal infection, caused by a viral, bacterial or fungal pathogen, respectively.

In case of an infectious disease, more specifically an infectious disease caused by a virus, the vaccine composition or kit of parts comprising a vaccine composition of the invention may be combined with therapies known to and applied by the skilled person in order to treat a subject suffering from a viral infection in order to improve clinical effectiveness of the respective therapy that said subject is receiving before, in parallel or after the vaccination to said subject, which can be any one or any combination of therapies with agents selected from antiviral agents like nucleoside analogues (e.g. Remdesivir, Favipiravir, Ribavirin, Tenofir Disoproxir Fumarate), neutralizing antibodies (e.g. from convalescent plasma), receptor decoys, membrane fusion inhibitors, viral and/or host protease inhibitors (e.g. Lopinavir/ritonavir), viral polymerases inhibitors (e.g. Naproxen), viral translation inhibitors, host-receptor antibodies, endocytosis inhibitors, kinase inhibitors (e.g. Baricitinib), lipidomic drugs, interferons, and corticosteroids (e.g. Dexamethasone).

In case of an infectious disease, more specifically an infectious disease caused by a bacterium, the vaccine composition or kit of parts comprising a vaccine composition of the invention may be combined with therapies known to and applied by the skilled person in order to treat a subject suffering from an bacterial infection in order to improve clinical effectiveness of the respective therapy that said subject is receiving before, in parallel or after the vaccination to said subject, which can be any one or any combination of therapies (or therapies with agents) selected from antibacterial agents like antibiotics against gram-negative and/or gram-positive bacteria strains like Aminoglycosides (such as Gentamicin, Streptomycin, Neomycin), Amphenicols (such as Chloramphenicol), Carbapenems (such as Imipenem, Meropenem), Cephalosporins (such as Cephalexin, Cefepime, Ceftaroline), Fluoroquinolones (such as Norfloxacin, Ciprofloxacin, Ofloxacin, Levofloxacin, Moxifloxacin, Gemifloxacin), Glycopeptides (such as Vancomycin, Teicoplanin, Bacitracin), lipoglycopeptides (such as Vancomycin, Daptomycin), Macrolides (such as Erythromycin, Azithromycin, Clarithromycin), Monobactams (Aztreonam), Oxazolidinones (such as Linezolid, Tedizolid), Penicillins (such as Amoxicillin, Floxacillin), Polymyxins (such as Polymyxin B, Polymyxin E), Rifamycins (such as Rifampin/Rifampicin, Rifabutin, Rifaximin, Rifapentine), Sulfonamides (such as Sulfamethoxazole, Sulfadiazine, Sulfisoxazole), Streptogramins (such as Quinupristin, Dalfopristin), and/or Tetracyclines (such as Doxycycline, Tetracycline), phage therapy (e.g. Biophage-PA), agents blocking bacterial virulence factors such as bacterial toxins (Raxibacumab, Shigamab Bezlotoxumab, MEDI4893), probiotics (such as Saccharomyces boulardii, Lactobacillus) and fecal or other microbiota transplantation.

In case of an infectious disease, more specifically an infectious disease caused by a fungus, the vaccine composition or kit of parts comprising a vaccine composition of the invention may be combined with therapies known to and applied by the skilled person in order to treat a subject suffering from a fungal infection in order to improve clinical effectiveness of the respective therapy that said subject is receiving before, in parallel or after the vaccination to said subject, which can be any one or any combination of therapies with agents selected from antimycotic agents like such inhibiting 1,3-β-d-glucan synthase responsible for the synthesis of β-d-glucans located in the fungal cell wall (i.e. Echinocandins such as Caspofungin, Micafungin, Anidulafungin), lanosterol 14-α-demethylase inhibitors (i.e. triazoles like Fluconazole, Itraconazole, Voriconazole, Posaconazole, and/or Isavuconazole), protein and DNA biosynthesis inhibitors (such as the pyrimidine analogue 5-Flucytosine (5-FC)), agents sequestrating ergosterol (which is the major part of fungal cell membrane), or inhibiting ergosterol biosynthesis (i.e. polyenes such as Amphotericin B), and/or siderophore biosynthesis enzyme inhibitors (Siderophores).

In a further embodiment, the present invention relates to a method for preparing a subject-specific vaccine composition for use in cancer therapy. Said method comprises the steps of:
(a) obtaining tumour and normal DNA or tumour and normal RNA sequence information for a subject diagnosed with cancer;
(b) identifying subject-specific MHC I and MHC II restricted tumour neoantigens based on the sequence information obtained in (a);
(c) determining the sequence of a first peptide, wherein the first peptide is an MHC I antigen and its sequence comprises a sequence comprising a subject-specific tumour neoantigen identified in step (b);
(d) determining the sequence of a corresponding second peptide, wherein the second peptide is an MHC II antigen, its sequence comprises a sequence comprising a subject-specific tumour neoantigen identified in step (b) and comprises the sequence of the first peptide;
(e) preparing the first peptide or a nucleic acid encoding the first peptide that can be expressed upon administration of said nucleic acid to a subject;
(f) preparing the second peptide or a nucleic acid encoding the second peptide that can be expressed upon administration of said nucleic acid to a subject; and
(g) formulating the subject-specific vaccine composition using the preparations of (e) and (f).

It is to be understood that in step (b), identifying subject-specific MHC I and MHC II restricted tumour neoantigens based on the sequence information obtained in (a) preferably means identifying subject-specific tumour-mutations and subject-specific MHC I and MHC II restricted tumour neoantigens based on the sequence information obtained in (a).

In a first step of the method for preparing a subject-specific vaccine composition for use in cancer therapy, i.e. in step (a) of said method, tumour and normal DNA or tumour and normal RNA sequence information for a subject diagnosed with cancer is obtained. The methods of sequencing subject's genome are known to the skilled person. For example, upon obtaining DNA or RNA samples from a subject, a state-of-the-art massive parallel sequencing methods (“next-generation sequencing”) such as those marketed by Roche (454 technology), Illumina (Solexa technology), ABI (Solid technology), Oxford Nanopore (nanopore sequencing) or Pacific Biosciences (SMRT technology) can be used. Preferably, DNA sequence information, preferably whole exome sequencing (WES) or whole genome sequencing (WGS) information for a subject's tumour and corresponding healthy tissue (preferably blood) is obtained.

As understood herein, the sequence information may be specific to the tumour and corresponding healthy tissue (preferably blood). Thus, step (a) of the method of the present invention preferably involves obtaining DNA or RNA sequence information, preferably DNA sequence information, and most preferably WES or WGS information from a sample of a tumour (e.g. obtained from a biopsy) and a corresponding healthy tissue (preferably blood) obtained from a subject.

In step (b) of the method for preparing a subject-specific vaccine composition for use in cancer therapy of the present invention, subject-specific and MHC I and MHC II-restricted tumour neoantigens are identified based on the sequence information obtained in (a). For this purpose, the patient's HLA type (all MHC I and MHC II alleles of that patient) is determined from the DNA sequencing data of the healthy normal tissue, preferably from WES or WGS data. The tumour-specific mutations are identified by comparing DNA or RNA sequences of the tumour and normal tissue, preferably from WES or WGS data. All potential MHC I (length of 8-13 amino acid residues) and MHC II peptides (length of 14-35 amino acid residues), which can be derived from each expressed tumour-specific mutation are determined, which may also be referred to as MHC I and MHC II restricted tumour neoantigens, respectively. Bioinformatic tools are used to predict which of those mutation-harbouring peptides may bind to the MHC (HLA) molecules of the patient with high affinity and/or which mutation-harbouring peptides may be presented by MHC (HLA) molecules of the patient with high likelihood. Finally, such mutation-harbouring peptides are filtered and/or prioritized according to specified criteria. Particularly preferred may be peptides derived from driver mutations. Further prioritization criteria may be, but are not limited to a high predicted MHC (HLA) presentation likelihood, a high predicted MHC (HLA) binding affinity, a high predicted processing likelihood, a high predicted immunogenicity, a high allele frequency and/or a high expression level of the respective variant in the tumour. An exemplary method for ranking and/or selecting subject-specific tumour neoantigens is described in WO 2020/043805.

In step (c) of the method for preparing a subject-specific vaccine composition for use in cancer therapy of the present invention, a sequence information for the first peptide is determined. As it is to be understood herein, the first peptide is designed to be a CD8 antigen. In other words, the first peptide is designed to be an MHC I antigen, i.e. be presentable by MHC I complex to CD8+ T-cells. The first peptide is, according to the invention, to be derived from one of the subject-specific tumour neoantigens identified (and prioritized) in step (b) of the method for preparing a subject-specific vaccine composition for use in cancer therapy of the present invention. In other words, the sequence of the first peptide comprises the sequence of a subject-specific tumour neoantigen identified (and prioritized) in step (b) of the method for preparing a subject-specific vaccine composition for use in cancer therapy of the present invention.

In step (d) of the method for preparing a subject-specific vaccine composition for use in cancer therapy of the present invention, a sequence information for the second peptide is determined. As it is to be understood herein, the second peptide is designed to be a CD4 antigen. In other words, the second peptide is designed to be an MHC II antigen, i.e. be presentable by MHC II complex to CD4+ T cells. The sequence of the second peptide comprises the sequence of the first peptide, as described hereinabove. Furthermore, the second peptide is, according to the invention, derived from one of the subject-specific tumour neoantigens identified (and prioritized) in step (b) of the method for preparing a subject-specific vaccine composition for use in cancer therapy of the present invention. In other words, the sequence of the second peptide comprises the sequence of one of the subject-specific tumour neoantigens identified (and prioritized) in step (b) of the method for preparing a subject specific vaccine composition for use in cancer therapy of the present invention.

Preferably, in the method of the present invention, the second peptide is so selected that its sequence comprises more than one, preferably two, three, four, five or more than five sequences of an MHC I antigen, one of which is the sequence of the first peptide. As mentioned before, the presence of sequences within the second peptide corresponding to MHC I antigen can be verified by the skilled person, for example with the use of bioinformatics prediction tools as described herein.

As it is preferably understood, the second peptide comprising more than one MHC I antigen (i.e. the second peptide for which more than one suitable first peptide, as provided in the present invention, exists or in other words can be selected) is preferred over the second peptide that does not include more than one MHC I antigen sequence. Furthermore, second peptides comprising five or more MHC I antigen sequences are most preferred.

In step (e) of the method for preparing a subject-specific vaccine composition for use in cancer therapy of the present invention the first peptide or a nucleic acid encoding the first peptide that can be expressed upon administration of said nucleic acid to a subject is prepared. Peptides are prepared according to the methods known to the skilled person, in particular according to the methods of the solid phase peptide synthesis, which have been described herein. Nucleic acids can be prepared according to the methods known to that skilled in the art, in particular by the methods of the solid phase nucleic acid synthesis or by molecular biology methods like in vitro transcription.

In step (f) of the method for preparing a subject-specific vaccine composition for use in cancer therapy of the present invention the second peptide or a nucleic acid encoding the second peptide that can be expressed upon administration of said nucleic acid to a subject is prepared according to but not limited to the methods described for step (e).

In step (g) of the method for preparing a subject-specific vaccine composition for use in cancer therapy of the present invention, the subject-specific vaccine composition is formulated using the preparations of (e) and (f). Preferably the subject-specific vaccine composition is formulated in a process comprising the step of mixing together the preparations of (e) and (f).

As understood to the skilled person, further excipient(s), carrier(s), adjuvant(s) and/or diluent(s) may be used in the process of formulating a vaccine composition, as described in detail hereinabove.

Preferably, in step (e) of the method for preparing a subject-specific vaccine composition for use in cancer therapy of the present invention, the first peptide is prepared in a form of a peptide or a pharmaceutically acceptable salt thereof. However, in one embodiment of the present invention the first peptide is prepared in a form of a nucleic acid encoding the first peptide that can be expressed upon administration of said nucleic acid to a subject. In another embodiment of the present invention the first peptide is prepared in a form of a vector (viral, bacterial, fungal/yeast) encoding the first peptide that can be expressed upon administration of said vector to a subject.

Preferably, in step (f) of the method for preparing a subject-specific vaccine composition for use in cancer therapy of the present invention, the second peptide is prepared in a form of a peptide or a pharmaceutically acceptable salt thereof. However, in one embodiment of the present invention the second peptide that can be expressed upon administration of said nucleic acid to a subject. In another embodiment of the present invention the second peptide is prepared in a form of a vector (viral, bacterial, fungal/yeast) encoding the second peptide that can be expressed upon administration of said vector to a subject.

In one embodiment of the method for preparing a subject-specific vaccine composition for use in cancer therapy of the present invention, in steps (e) to (g) both the first and the second peptide are prepared in a form of one nucleic acid encoding both the first and the second peptide and that can be expressed upon administration of said nucleic acid to a subject. The first and the second peptide can also be prepared in a form of a vector (viral, bacterial, fungal/yeast) encoding both the first and the second peptide that can be expressed upon administration of said vector to a subject.

Preferably, the sequence of the first peptide is so determined in step (c) of the method for preparing a subject-specific vaccine composition for use in cancer therapy of the present invention, that the sequence of the first peptide is 8 to 13 amino acid residues long.

Preferably, the sequence of the second peptide is so determined in step (d) of the method for preparing a subject-specific vaccine composition for use in cancer therapy of the present invention, that the sequence of the second peptide is at least 14 amino acid residues long. More preferably, the sequence of the second peptide is so determined in step (d) of the method for preparing a subject-specific vaccine composition for use in cancer therapy of the present invention, that the sequence of the second peptide is 14 to 35 amino acid residues long. Even more preferably, the sequence of the second peptide is so determined in step (d) of the method for preparing a subject-specific vaccine composition for use in cancer therapy of the present invention, that the sequence of the second peptide is 14 to 25 amino acid residues long.

Preferably, in the method for preparing a subject-specific vaccine composition for use in cancer therapy of the present invention, the first peptide is determined to be an MHC I antigen by using a bioinformatic prediction algorithm.

Preferably, in the method for preparing a subject-specific vaccine composition for use in cancer therapy of the present invention, the second peptide is determined to be an MHC II antigen by using a bioinformatic prediction algorithm.

Exemplary prediction algorithms for performing said predictions are as described hereinabove.

In a further embodiment, the present invention relates to a method of inducing an immune response in a subject. Said method comprises the step of administering the vaccine composition as described hereinabove, or the parts of the kit of parts of the present invention as described hereinabove, to a subject in need thereof, preferably to a human subject in need thereof. It will be understood that an effective amount for inducing said immune response of the vaccine composition of the invention or the parts of the kit of parts of the invention is to be administered in accordance with this method. Effective amounts of the first peptide and the second peptide as described herein are discussed hereinabove. Furthermore, exemplary effective amounts of the first peptide and the second peptide are derivable from the exemplary embodiments of the present invention as illustrated in the Examples section. Typically, as encompassed by the present invention, the first peptide and the second peptide are each dosed in an amount of between 1 μg and 1000 μg per dose, preferably in an amount of at least 10 μg, at least 20 μg, at least 50 μg or at least 100 μg, and also preferably in an amount not exceeding 800 μg, not exceeding 600 μg or not exceeding 500 μg. More preferably, the first peptide and the second peptide are each dosed in an amount of between 300 μg and 500 μg. In a preferred but non-limiting example, each first peptide and second peptide are dosed at a dose of about 400 μg each, preferably at a dose of 400 μg each.

In a further embodiment, the present invention relates to a method of increasing an immune response in a subject. Said method comprises the step of administering the vaccine composition as described hereinabove, or the parts of the kit of parts of the present invention as described hereinabove, to a subject in need thereof, preferably to a human subject in need thereof. It will be understood that an effective amount of the vaccine composition of the invention or the parts of the kit of parts of the invention for increasing said immune response is to be administered in accordance with this method. Effective amounts of the first peptide and the second peptide as described herein are discussed hereinabove. Furthermore, exemplary effective amounts of the first peptide and the second peptide are derivable from the exemplary embodiments of the present invention as illustrated in the Examples section.

In one embodiment, in the method of inducing an immune response in a subject of the present invention or a method of increasing an immune response in a subject of the present invention, said immune response is an immune response against an infectious pathogen. Infectious pathogen and an infectious disease caused by such pathogen as defined herein is not particularly limited. It can be a virus leading to a viral infectious disease, a bacteria leading to a bacterial infectious disease, or a fungus leading to a fungal infectious disease. Thus, in one embodiment, the method of inducing an immune response in a subject of the present invention or a method of increasing an immune response in a subject of the present invention relates to a method of preventing or treating an infectious disease in a subject. Preferably, said infectious disease is a viral infectious disease, a bacterial infectious disease, or a fungal infectious disease. In other words, preferably said infectious disease is caused by a viral infection, a bacterial infection or a fungal infection.

Preferably, in the method of inducing an immune response in a subject of the present invention or a method of increasing an immune response in a subject of the present invention, said immune response is an immune response against a tumour present in said subject. Thus preferably, the method of inducing an immune response in a subject of the present invention or a method of increasing an immune response in a subject of the present invention relates to a method of preventing or treating a cancer disease in a subject.

In a further embodiment, the present invention relates to use of a first peptide of the present invention or a nucleic acid encoding the first peptide that can be expressed upon administration of said nucleic acid to a subject, as described herein; and a second peptide of the present invention or a nucleic acid encoding the second peptide that can be expressed upon administration of said nucleic acid to a subject as described herein, for a manufacture of a vaccine composition for use in a method of inducing an immune response in a subject.

In a further embodiment, the present invention relates to use of a first peptide of the present invention or a nucleic acid encoding the first peptide that can be expressed upon administration of said nucleic acid to a subject, as described herein; and a second peptide of the present invention or a nucleic acid encoding the second peptide that can be expressed upon administration of said nucleic acid to a subject as described herein, for a manufacture of a vaccine composition for use in a method of increasing an immune response in a subject.

In a further embodiment, the present invention relates to use of a first peptide of the present invention or a nucleic acid encoding the first peptide that can be expressed upon administration of said nucleic acid to a subject, as described herein; and a second peptide of the present invention or a nucleic acid encoding the second peptide that can be expressed upon administration of said nucleic acid to a subject as described herein, for a manufacture of a vaccine composition for use in a method for treating or preventing a cancer disease in a subject.

The invention will be illustrated through examples, which however should not be construed as limiting.

### EXAMPLES

### Vaccine Formulation & Administration

Peptides were synthesized by Solid-Phase Peptide Synthesis (SPPS). Lyophilized peptides (HCl salt) were dissolved in dimethyl sulfoxide (DMSO), mixed, and sterile filtered. DMSO concentration was adjusted to 33% with water. The final concentration of the multipeptide solution was 0.8 mg/ml per peptide.

Per vaccination, 0.5 ml peptide solution (i.e. 400 μg of each peptide/dose) were injected intracutaneously in the left or right lower abdomen followed by subcutaneous injection of 42-83 μg sargramostim and/or topical application of 6.25-12.5 mg imiquimod (in form of a cream) in the same area. The patient was vaccinated four times during the priming phase (first month) of the vaccination schedule with subsequent boost vaccinations every four to eight weeks.

### Immune Monitoring

Immune monitoring (IMM) was performed for detection of vaccine-induced T-cell responses. Peripheral blood mononuclear cells (PBMCs) were isolated from whole blood using Biocoll Separation Solution (Biochrom). After density gradient centrifugation, PBMCs were washed and cryopreserved in freezing medium containing 10% DMSO (VWR) until further usage. After thawing, PBMCs were cultivated for 12 hours in TexMACS medium (Miltenyi) containing 3 μg/ml DNAse I (Sigma-Aldrich). After pre-incubation, cells were washed and re-sowed in TexMACS medium containing 1% Penicillin-Streptomycin (Sigma-Aldrich). Individually predicted, neoantigen-derived peptides (synthesized by EMC microcollections GmbH) were added at a concentration of 1 μg/ml for MHC class I peptides and 5 μg/ml for MHC class II peptides. Cells were cultivated in presence of peptides for 12 days. After the first 24 hours of cultivation, 10 U/ml IL-2 (Miltenyi Biotec) and 10 ng/ml IL-7 (Miltenyi Biotec) were added. Medium was changed every 2-3 days. After 12 days of cultivation, expanded cells were restimulated with corresponding peptides at the same concentration and additionally incubated for 14 hours in presence of Golgi inhibitors (Golgi Plug; BD biosciences; concentration: 1 μl/ml;).

The readout was Flow Cytometric Analysis after Intracellular Cytokine Staining (ICS). After cultivation, cells were washed and stained extra- and intracellularly using fluorochrome-conjugated antibodies titrated to their optimal concentrations. Finally, cells were measured on a Novocyte 3005R cytometer (Agilent).

Data were analyzed using FlowJo version 10.5.3 (FlowJo LLC). Briefly, CD4+ and CD8+ T-cells were gated within viable CD3+ lymphocytes and analyzed separately for each functional marker (CD154, IFN-γ, TNF, IL-2). Peptide-specific responses were evaluated using the stimulation index (SI). The stimulation index is the calculated ratio of polyfunctional activated CD4+ or CD8+ T-cells (positive for at least two T cell activation markers including CD154, IFN-γ, TNF, and/or IL-2) in the peptide-stimulated sample to the negative control sample (DMSO). Additionally, a minimal frequency of 0.1% of reactive T-cells positive for at least one T cell activation marker including CD154, IFN-γ, TNF and/or IL-2 had to be reached among a minimum of 10,000 measured CD4+ or CD8+ events. Vaccine-induced immune responses were categorized as follows: SI≥2: weak response (+), SI≥3: positive response (++), SI≥5: strong response (+++), SI≥10: very strong response (++++).

### Example 1

In total, immunogenicity data for 1066 neoantigen peptides from 100 cancer patients were collected. Before further analysis data were filtered:
Peptides from patients with less than 4 vaccinations were excluded, since it requires a few vaccinations to induce a T cell response
Patients having no vaccine peptide with a T cell response were excluded, since it is unclear whether lack of immune response is due to the vaccinated epitopes or the overall immune status of that patient. Anti-cancer therapy and overall health condition may have generally weakened the immune system of the patient, preventing any T cell response to vaccinated epitopes.
For some patients the amount of PBMC obtained from a blood draw was insufficient to analyse all vaccinated peptides separately for immune monitoring. In such cases peptides were pooled and incubated with PBMCs for analysis. However, peptides analysed in pools were generally excluded from the analysis presented here, since it is unclear which peptide in a pool may be responsible for a T cell response. Since more often short peptides were pooled there was a higher number of co-vaccinated MHC II epitopes (87) available for analysis than co-vaccinated MHC I epitopes (77).

After filtering IMM data of 262 long MHC class II peptides from 71 patients and 380 short MHC class I peptides from 67 patients were included in the analysis.

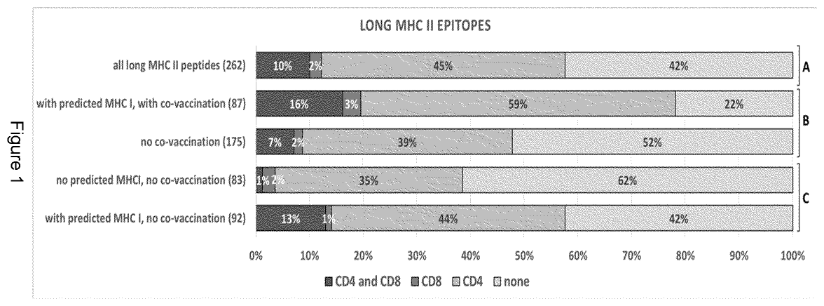

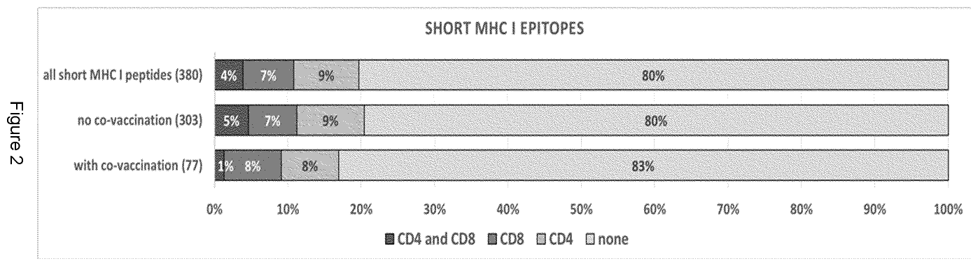

Within this cohort, an induced immune response was observed for 58% of all tested long peptides (FIG. 1A) and for 20% of all tested short peptides (FIG. 2) when all peptide-activated immune cell populations were merged (namely those with solely CD4+, solely CD8+ or both CD4+ as well as CD8+ T-cell responses)

Next, induced immune responses against long MHC II restricted peptides were analysed discriminating between those co-vaccinated with a corresponding short MHC I-restricted peptide and those for which only a long MHC II peptide was vaccinated against a given mutation. For the long and short peptide pairs, an induced immune response to 78% of long peptides was observed. In comparison, an induced immune response to only 48% of long peptides which were vaccinated alone was observed (FIG. 1B).

For those long MHC II restricted peptides which were injected without the corresponding short MHC I restricted peptide, induced immune responses to long peptides bearing a predicted short MHC I epitope versus those long peptides not bearing a predicted short MHC I epitope were compared next. For the long peptides including a short MHC I epitope, an induced immune response in 58% of cases was observed. In comparison, an induced immune response was observed in only 38% of cases where only the long peptide without a short epitope was vaccinated (FIG. 1C).

In contrast co-vaccination of a matched long MHC II-restricted peptide did not lead to a similar increase in immune responses to short MHC I restricted peptides (FIG. 2).

### Example 1.1

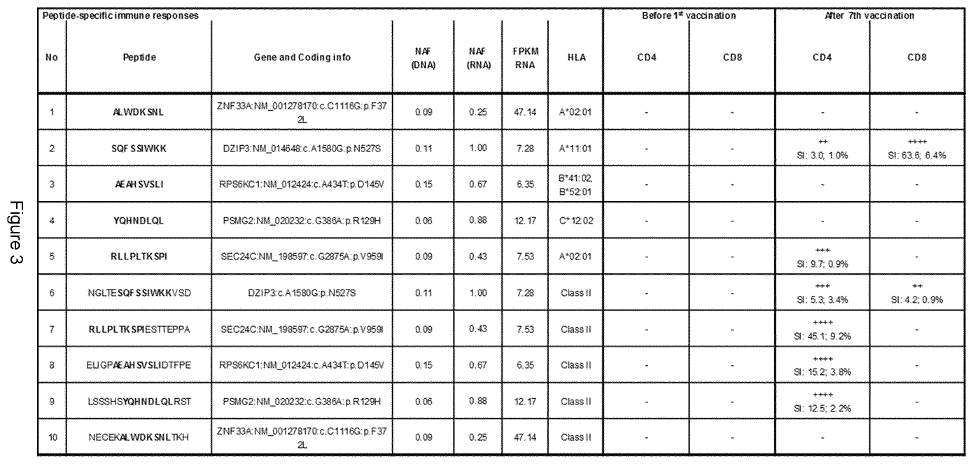

Paired neoantigen peptides for 5 of 5 targeted somatic mutations were co-vaccinated, i.e. administered to a subject as corresponding MHC I antigen and MHC II antigen. As shown in the Table presented in FIG. 3, several strong CD4+ and CD8+ T-cell responses were observed.

### Example 1.2

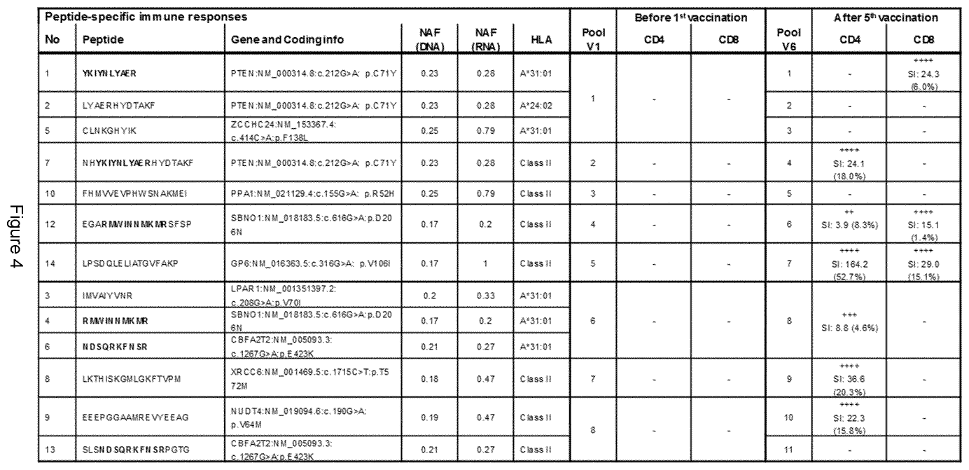

Paired neoantigen peptides for 3 of 9 targeted somatic mutations were co-vaccinated, i.e. administered to a subject as corresponding MHC I antigen and MHC II antigen. In this case two MHC I and one MHC II antigens were vaccinated for the PTEN mutant. For all other mutants one MHC I and one MHC II antigen were co-applied. As shown in the Table presented in FIG. 4, several strong CD4+ and CD8+ T-cell responses were observed against neoepitopes vaccinated in pairs and alone.

### Example 1.3

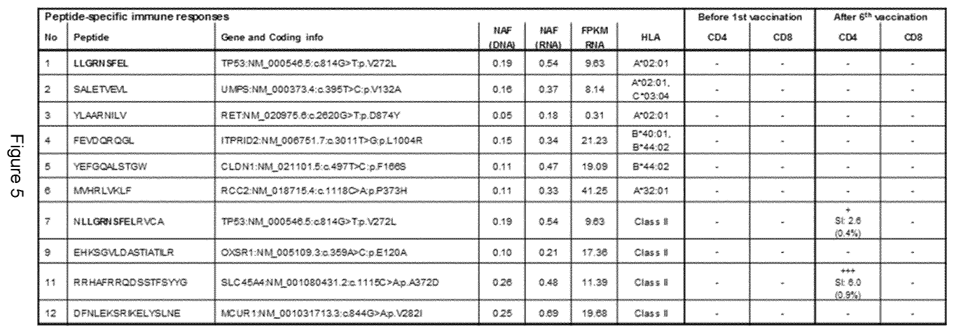

Paired neoantigen peptides for 1 of 9 targeted somatic mutations were co-vaccinated, i.e. administered to a subject as corresponding MHC I antigen and MHC II antigen. As shown in the Table presented in FIG. 5, no CD8+ T-cell responses were observed, but two moderate CD4+ T-cell responses against a paired and an unpaired MHC II neoantigen.

### Example 1.4

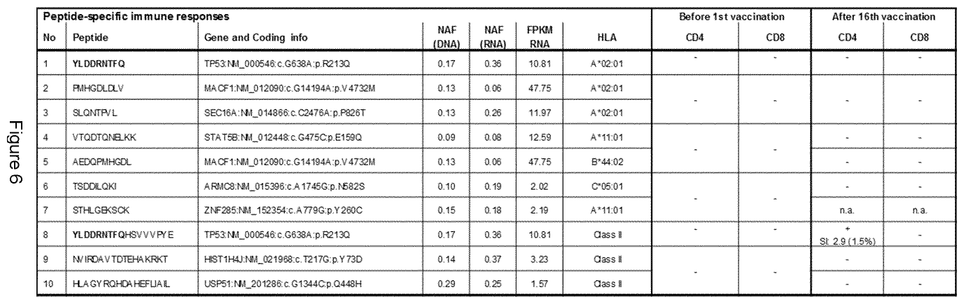

Paired neoantigen peptides for 1 of 9 targeted somatic mutations were co-vaccinated, i.e. administered to a subject as corresponding MHC I antigen and MHC II antigen. As shown in the Table presented in FIG. 6, a single CD4+ T-cell response against the paired MHC II neoantigen and no CD8+ T-cells responses were observed.

### Example 1.5

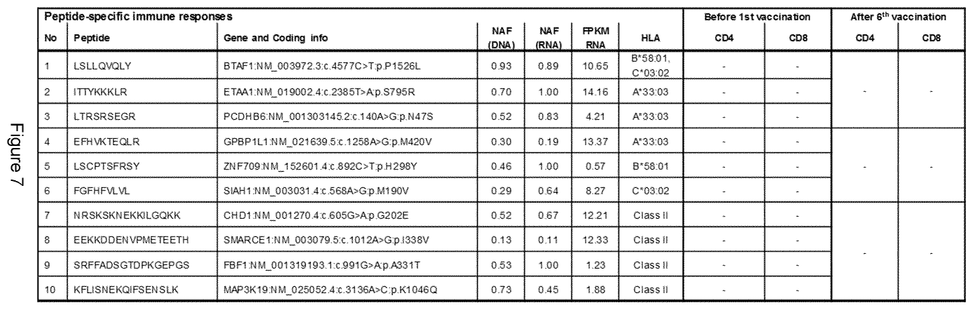

For none of 10 targeted somatic mutations paired neoantigen peptides were co-vaccinated, i.e. administered to a subject as corresponding MHC I antigen and MHC II antigen. As shown in the Table presented in FIG. 7, no immune responses were observed.

In all examples 1.1. to 1.5 there were no pre-existing T-cell responses detected prior to the vaccination regime (baseline) indicating that in all observed cases the vaccination induced a de novo immune response.

Tables display the amino acid sequence of the vaccinated peptide as well as the mutated gene. NAF =Novel allele frequency: frequency with which the mutated allele was occurring in the tumour sequencing. Neoantigen-specific CD4+ or CD8+ T-cells are detected before therapy and after the priming phase. SI: Stimulation index, ratio of polyfunctional activated CD4+ or CD8+ T-cells (positive for at least two T cell activation markers including CD154, IFN-γ, TNF and/or IL-2) in the peptide-stimulated sample compared to the unstimulated control. Additionally, the percentage of activated CD4+ or CD8+ T-cells (positive for at least one activation T cell marker including CD154, IFN-γ, TNF and/or IL-2) above background and after in vitro amplification is given. The percentages do not directly reflect the frequencies in vivo.

### Example 2

For Example 2 a subset of tumour-mutation derived neoantigen epitopes from Example 1 was independently analysed including 317 representative MHC I epitopes and 139 representative MHC II epitopes. For this analysis the number of predicted MHC I epitopes included in the MHC II epitopes were determined by the three MHC I epitope prediction algorithms NetMHC, NetMHCpan and SYFPEITHI. The results are described in the following Examples 2.1-2.5.

### Example 2.1

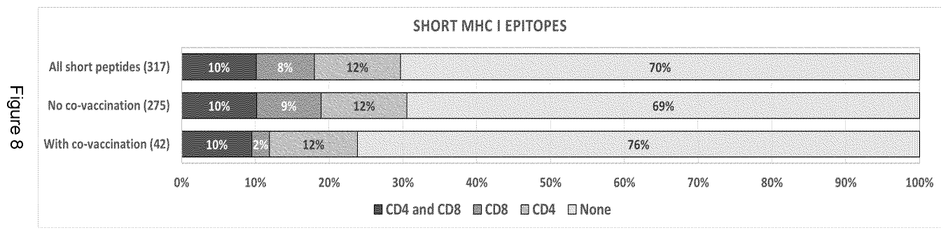

The frequencies of CD4+ and CD8+ T cell responses to 317 short MHC I epitopes derived from tumour mutations and vaccinated into cancer patients together with other neoantigen peptides were determined. For this analysis MHC I epitopes were discriminated according to the co-vaccination status (FIG. 8). Frequencies of T cell responses were determined by incubating PBMCs of the respective patient with the short MHC I peptide epitope and by assessing the proportion (%) of peptide-activated T cells using intracellular cytokine staining and FACS analysis. Epitopes were counted as immunogenic if the fraction of activated CD4+ or CD8+ T cells was >=0.1% and the stimulation index was >=2 (ratio of polyfunctional activated CD4+ or CD8+ T-cells in the peptide-stimulated sample to the negative control sample incubated with DMSO). The results indicated that co-vaccination of MHC I and MHC II pairs did not increase CD4+ or CD8+ T cell responses to MHC I epitopes, when compared with MHC I epitopes vaccinated without the corresponding MHC II epitope. Indeed, co-vaccinated MHC I epitopes showed a reduced frequency of CD8+ T cell responses compared to MHC I epitopes vaccinated alone (12% vs. 19%).

### Example 2.2

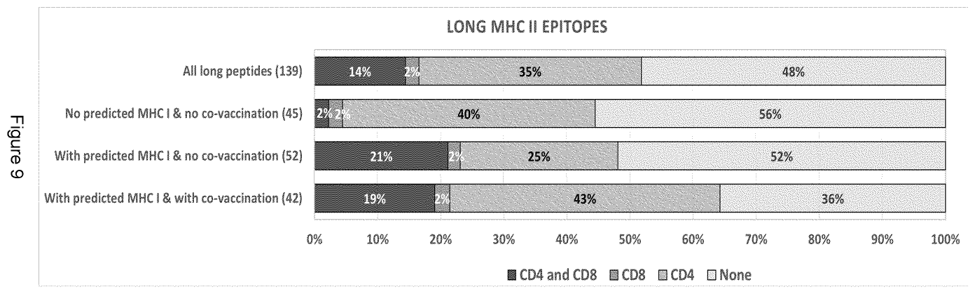

The frequencies of CD4+ and CD8+ T cell responses to 139 long MHC II epitopes derived from tumour mutations and vaccinated into cancer patients together with other neoantigen peptides were determined. For this analysis MHC II epitopes were discriminated according to the inclusion of any in silico predicted MHC I epitope (nested epitope) and co-vaccination status (FIG. 9). Frequencies of T cell responses were determined by incubating PBMCs of the respective patient with the long MHC II peptide epitope and by assessing the proportion (%) of peptide-activated T cells using intracellular cytokine staining and FACS analysis. Epitopes were counted as immunogenic if the fraction of activated CD4+ or CD8+ T cells was >=0.1% and the stimulation index was >=2. As expected, MHC II epitopes vaccinated without a corresponding MHC I epitope showed a vastly increased frequency of CD8+ T cell responses if they contained a predicted MHC I epitope compared to MHC II epitopes not containing a predicted MHC I epitope (23% vs. 4%). Also, as expected frequencies of CD4+ T cell activation remained similar in both groups (46% vs. 42%). Co-vaccination of MHC I and MHC II pairs mainly increased CD4+ T cell responses to MHC II epitopes when compared with MHC II epitopes which also contain a predicted MHC I epitope, but were vaccinated alone (62% vs. 46%). CD8+ T cell responses remained similar in both groups (21% vs. 23%).

### Example 2.3

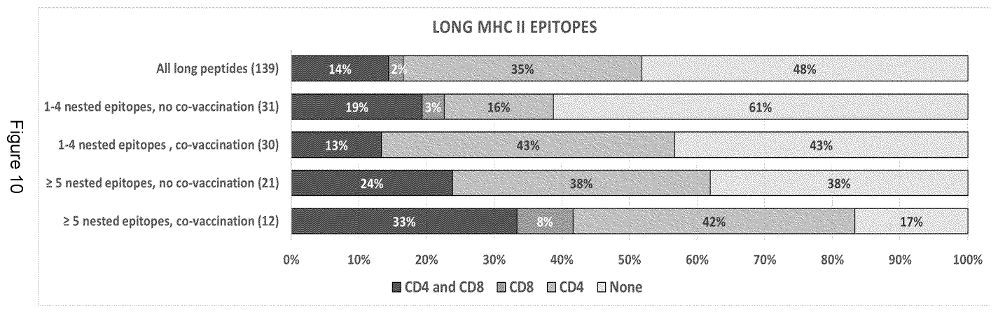

The frequencies of CD4+ and CD8+ T cell responses to 139 long MHC II epitopes derived from tumour mutations and vaccinated into cancer patients together with other neoantigen peptides were determined. For this analysis MHC II peptides were discriminated according to the number of included in silico predicted MHC I epitopes (nested epitopes) and co-vaccination status (FIG. 10). Frequencies of T cell responses were determined by incubating PBMCs of the respective patient with the long MHC II peptide epitope and by assessing the proportion (%) of peptide-activated T cells using intracellular cytokine staining and FACS analysis. Epitopes were counted as immunogenic if the fraction of activated CD4+ or CD8+ T cells was >=0.1% and the stimulation index was >=2. The results show that MHC II epitopes with more than 4 predicted MHC I epitopes had higher frequencies of CD4+ T cell responses compared to MHC II epitopes with 1-4 predicted MHC I epitopes. This observation was seen in both groups of MHC II epitopes, those which were co-vaccinated with a corresponding MHC I epitope and those which were not. However, MHC II epitopes containing more than 4 predicted MHC I epitopes which were co-vaccinated with one of these predicted MHC I epitopes showed by far the highest frequencies of CD4+ and CD8+ T cell responses (75% and 41%, respectively).

### Example 2.4

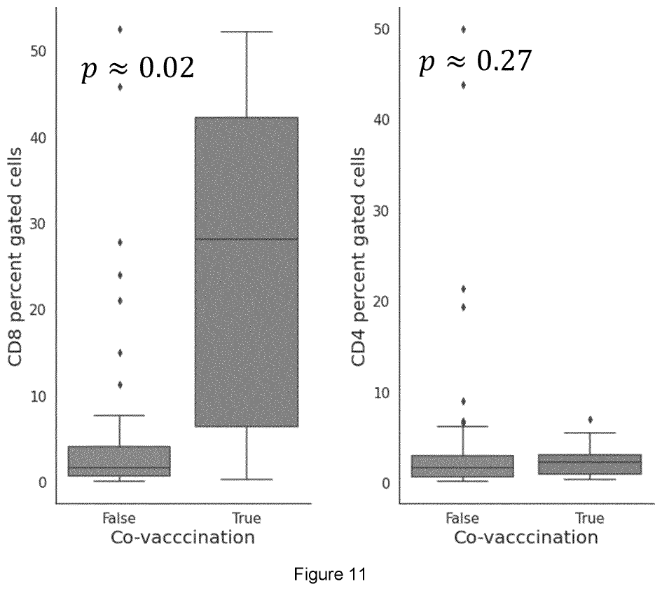

The strengths of CD8+ and CD4+ T cell responses to short MHC I epitopes were investigated. For this analysis the MHC I epitopes were discriminated according to the co-vaccination status (FIG. 11). T cell response strength was determined by incubating PBMCs of the respective patient with the respective short MHC I peptide epitope and by assessing the proportion (%) of peptide-activated T cells using intracellular cytokine staining and FACS analysis. Included were only epitopes with an immune response. While the strength of CD4+ T cell responses remained similar the strength of CD8+ T cell responses (% peptide-activated T cells) was significantly increased for immunogenic MHC I epitopes which were co-vaccinated with the corresponding MHC II epitope compared to immunogenic MHC I epitopes vaccinated alone. Provided p-values were obtained from Mann-Whitney testing without multiple test correction.

### Example 2.5

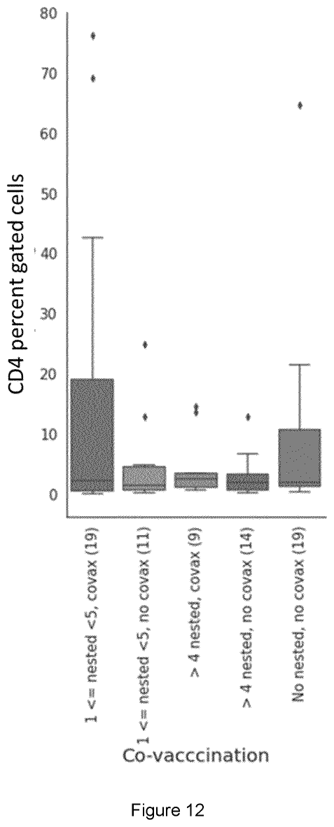

The strength of CD4+ T cell responses to long MHC II epitopes was investigated and epitopes were discriminated according to the number of included in silico predicted MHC I epitopes (nested epitopes) and co-vaccination status (FIG. 12). T cell response strength was determined by incubating PBMCs of the respective patient with the long MHC II peptide epitope and by assessing the proportion (%) of peptide-activated T cells using intracellular cytokine staining and FACS analysis. In this analysis only epitopes with an immune response were included. A trend for stronger CD4+ T cell responses to immunogenic MHC II epitopes was observed when these were co-vaccinated with the corresponding MHC I epitope and compared with immunogenic MHC II epitopes vaccinated alone. This observation was similar for the group of long MHC II peptides with 1-4 predicted MHC I epitopes and the group of long MHC II peptides with more than 4 predicted MHC I epitopes.

Further embodiments and/or examples of the present invention are provided in the following numbered items.
1. A vaccine composition comprising
   a first peptide or a nucleic acid encoding the first peptide that can be expressed upon administration of said nucleic acid to a subject, wherein the first peptide is an MHC I antigen; and
   a second peptide or a nucleic acid encoding the second peptide that can be expressed upon administration of said nucleic acid to a subject, wherein the second peptide is an MHC II antigen;
   wherein the sequence of the first peptide is comprised in the sequence of the second peptide.
2. A kit of parts comprising
   a vaccine composition comprising a first peptide or a nucleic acid encoding the first peptide that can be expressed upon administration of said nucleic acid to a subject, wherein the first peptide is an MHC I antigen; and
   a vaccine composition comprising a second peptide or a nucleic acid encoding the second peptide that can be expressed upon administration of said nucleic acid to a subject, wherein the second peptide is an MHC II antigen;
   wherein the sequence of the first peptide is comprised in the sequence of the second peptide.
3. The vaccine composition of item 1 or the kit of parts of item 2, wherein the first peptide is present in a form of a peptide or a pharmaceutically acceptable salt thereof.
4. The vaccine composition of item 1 or 3 or the kit of parts of item 2 or 3, wherein the first peptide is present in a form of a nucleic acid encoding the first peptide that can be expressed upon administration of said nucleic acid to a subject.
5. The vaccine composition of any one of items 1, 3 or 4, or the kit of parts of any one of items 2 to 4, wherein the second peptide is present in a form of a peptide or a pharmaceutically acceptable salt thereof.
6. The vaccine composition of any one of items 1 or 3 to 5 or the kit of parts of any one of items 2 to 5, wherein the second peptide is present in a form of a nucleic acid encoding the second peptide that can be expressed upon administration of said nucleic acid to a subject.
7. The vaccine composition of any one of items 1 or 3 to 6, or the kit of parts of any one of items 2 to 6, wherein the sequence of the first peptide is 8 to 13 amino acid residues long.
8. The vaccine composition of any one of items 1 or 3 to 7, or the kit of parts of any one of items 2 to 7, wherein the sequence of the second peptide is at least 14 amino acid residues long, preferably is 14 to 35 amino acid residues long.
9. The vaccine composition of any one of items 1 or 3 to 8, or the kit of parts of any one of items 2 to 8, wherein the first peptide is determined to be an MHC I antigen by using a bioinformatic prediction algorithm.
10. The vaccine composition of any one of items 1 or 3 to 9, or the kit of parts of any one of items 2 to 9, wherein the second peptide is determined to be an MHC II antigen by using a bioinformatic prediction algorithm.
11. The vaccine composition of any one of items 1 or 3 to 10, or the kit of parts of any one of items 2 to 10, wherein the vaccine composition is a cancer vaccine composition or wherein the vaccine compositions are cancer vaccine compositions.
12. The vaccine composition or the kit of parts of item 11, wherein the sequence of the first peptide is derived from a tumour neoantigen.
13. The vaccine composition or the kit of parts of item 11 or 12, wherein the sequence of the first peptide comprises a cancer-specific mutation, preferably a subject specific and tumour-specific mutation.
14. The vaccine composition or the kit of parts of any one of items 11 to 13, wherein the vaccine composition is tailored to a specific subject by the sequence of the first peptide being a sequence derived from a subject-specific tumour neoantigen.
15. The vaccine composition or the kit parts of any one of items 11 to 14, wherein the vaccine composition is tailored to a specific subject by the sequence of the first peptide comprising a subject-specific tumour mutation.
16. The vaccine composition of any one of items 1 or 3 to 15 or the kit of parts of any one of items 2 to 15 for use as a vaccine.
17. The vaccine composition of any one of items 1 or 3 to 15 or the kit of parts of any one of items 2 to 15 for use in a method of inducing an immune response in a subject.
18. The vaccine composition of any one of items 1 or 3 to 15 or the kit of parts of any one of items 2 to 15 for use in a method of increasing an immune response in a subject, preferably wherein the immune response is against an infectious disease or against cancer, preferably wherein the infectious disease is a viral infectious disease, a bacterial infectious disease or a fungal infectious disease.
19. The vaccine composition of any one of items 1 or 3 to 15 or the kit of parts of any one of items 2 to 15 for use in therapy.
20. The vaccine composition of any one of claims 1 or 3 to 15 or the kit of parts of any one of items 2 to 15 for use in cancer therapy.
21. A method for preparing a subject-specific vaccine composition for use in cancer therapy, the method comprising the steps of
   (a) obtaining tumour and normal DNA or tumour and normal RNA sequence information for a subject diagnosed with cancer;
   (b) identifying subject-specific MHC I and MHC II restricted tumour neoantigens based on the sequence information obtained in (a);
   (c) determining the sequence of a first peptide, wherein the first peptide is an MHC I antigen and its sequence comprises a sequence comprising a subject-specific tumour neoantigen identified in step (b);
   (d) determining the sequence of a corresponding second peptide, wherein the second peptide is an MHC II antigen, its sequence comprises a sequence comprising a subject-specific tumour neoantigen identified in step (b) and comprises the sequence of the first peptide;
   (e) preparing the first peptide or a nucleic acid encoding the first peptide that can be expressed upon administration of said nucleic acid to a subject;
   (f) preparing the second peptide or a nucleic acid encoding the second peptide that can be expressed upon administration of said nucleic acid to a subject; and
   (g) formulating the subject-specific vaccine composition using the preparations of (e) and (f).
22. The method of item 21, wherein the first peptide is prepared in a form of a peptide or a pharmaceutically acceptable salt thereof.
23. The method of item 21, wherein the first peptide is prepared in a form of a nucleic acid encoding the first peptide that can be expressed upon administration of said nucleic acid to a subject.
24. The method of any one of items 21 to 23, wherein the second peptide is prepared in a form of a peptide or a pharmaceutically acceptable salt thereof.
25. The method of any one of items 21 to 23, wherein the second peptide is prepared in a form of a nucleic acid encoding the second peptide that can be expressed upon administration of said nucleic acid to a subject.
26. The method of any one of items 21 to 25, wherein the sequence of the first peptide is 8 to 13 amino acid residues long.
27. The method of any one of items 21 to 26, wherein the sequence of the second peptide is at least 14 amino acid residues long, preferably 14 to 35 amino acid residues long.
28. The method of any one of items 21 to 27, wherein the first peptide is determined to be an MHC I antigen by using a bioinformatic prediction algorithm.
29. The method of any one of items 21 to 28, wherein the second peptide is determined to be an MHC I antigen by using a bioinformatic prediction algorithm.
30. A method of inducing an immune response in a subject, the method comprising administering the vaccine composition of any one of items 1 or 3 to 15 or the parts of the kit of parts of any one of items 2 to 15 to a subject in need thereof.
31. A method of increasing an immune response in a subject, the method comprising administering the vaccine composition of any one of items 1 or 3 to 15 or the parts of the kit of parts of any one of items 2 to 15 to a subject in need thereof.
32. A method of treating a cancer disease in a subject, the method comprising administering the vaccine composition of any one of items 11 to 15 or the parts of the kit of parts of any one of items 11 to 15 to a subject in need thereof.
33. Use of:
   a first peptide or a nucleic acid encoding the first peptide that can be expressed upon administration of said nucleic acid to a subject as described in any one of items 1 to 15; and
   a second peptide or a nucleic acid encoding the second peptide that can be expressed upon administration of said nucleic acid to a subject as described in any one of items 1 to 15;
   for a manufacture of a vaccine composition for use in a method of inducing an immune response in a subject.
34. Use of:
   a first peptide or a nucleic acid encoding the first peptide that can be expressed upon administration of said nucleic acid to a subject as described in any one of items 1 to 15; and
   a second peptide or a nucleic acid encoding the second peptide that can be expressed upon administration of said nucleic acid to a subject as described in any one of items 1 to 15;
   for a manufacture of a vaccine composition for use in a method of increasing an immune response in a subject.
35. Use of:
   a first peptide or a nucleic acid encoding the first peptide that can be expressed upon administration of said nucleic acid to a subject as described in any one of items 1 to 15; and
   a second peptide or a nucleic acid encoding the second peptide that can be expressed upon administration of said nucleic acid to a subject as described in any one of items 1 to 15;
   for a manufacture of a vaccine composition for use in a method of treating or preventing a cancer disease in a subject.

Further embodiments and/or examples of the invention are disclosed in the following numbered paragraphs:
1. A vaccine composition comprising
   a first peptide or a nucleic acid encoding the first peptide that can be expressed upon administration of said nucleic acid to a subject, wherein the first peptide is an MHC-I antigen; and
   a second peptide or a nucleic acid encoding the second peptide that can be expressed upon administration of said nucleic acid to a subject, wherein the second peptide is an MHC-II antigen;
   wherein the sequence of the first peptide is comprised in the sequence of the second peptide.
2. A kit of parts comprising
   a vaccine composition comprising a first peptide or a nucleic acid encoding the first peptide that can be expressed upon administration of said nucleic acid to a subject, wherein the first peptide is an MHC-I antigen; and
   a vaccine composition comprising a second peptide or a nucleic acid encoding the second peptide that can be expressed upon administration of said nucleic acid to a subject, wherein the second peptide is an MHC-II antigen;
   wherein the sequence of the first peptide is comprised in the sequence of the second peptide.
3. The vaccine composition of paragraph 1 or the kit of parts of paragraph 2,
   wherein the first peptide is present in a form of a peptide or a pharmaceutically acceptable salt thereof, and/or
   wherein the second peptide is present in a form of a peptide or a pharmaceutically acceptable salt thereof.
4. The vaccine composition of paragraphs 1 or 3, or the kit of parts of paragraph 2 or 3, wherein the sequence of the first peptide is 8 to 13 amino acid residues long, and/or
   wherein the sequence of the second peptide is at least 14 amino acid residues long, preferably is 14 to 35 amino acid residues long.
5. The vaccine composition of any one of paragraphs 1, 3 or 4, or the kit of parts of any one of paragraphs 2 to 4,
   wherein the first peptide is determined to be an MHC-I antigen by using a bioinformatic prediction algorithm, and/or
   wherein the second peptide is determined to be an MHC-II antigen by using a bioinformatic prediction algorithm.
6. The vaccine composition of any one of paragraphs 1 or 3 to 5, or the kit of parts of any one of paragraphs 2 to 5, wherein the vaccine composition is a pathogen vaccine composition or wherein the vaccine compositions are pathogen vaccine compositions.
7. The vaccine composition of any one of paragraphs 1 or 3 to 5, or the kit of parts of any one of claims 2 to 5, wherein the vaccine composition is a cancer vaccine composition or wherein the vaccine compositions are cancer vaccine compositions.
8. The vaccine composition or the kit of parts of paragraph 7, wherein the sequence of the first peptide is derived from an unmutated tumour-associated antigen.
9. The vaccine composition or the kit of parts of paragraph 7,
   wherein the sequence of the first peptide is derived from a tumour neoantigen, and/or
   wherein the sequence of the first peptide comprises a cancer-specific mutation, preferably a subject specific and tumour-specific mutation, and/or
   wherein the vaccine composition is tailored to a specific subject by the sequence of the first peptide being a sequence derived from a subject-specific tumour neoantigen, and/or
   wherein the vaccine composition is tailored to a specific subject by the sequence of the first peptide comprising a subject-specific tumour-specific mutation.
10. The vaccine composition of any one of paragraphs 1 or 3 to 9 or the kit of parts of any one of claims 2 to 9 for use as a vaccine.
11. The vaccine composition of paragraph 10, wherein the vaccine is a prophylactic vaccine applied before the onset of a disease.
12. The vaccine composition of any one of paragraphs 1 or 3 to 9 or the kit of parts of any one of paragraphs 2 to 9 for use in a method of inducing an immune response in a subject or for use in a method of increasing an immune response in a subject.
13. The vaccine composition of any one of paragraphs 1 or 3 to 5 or 7 to 9 or the kit of parts of any one of paragraphs 2 to 5 or 7 to 9 for use in therapy, preferably for use in cancer therapy.
14. A method for preparing a subject-specific vaccine composition for use in cancer therapy, the method comprising the steps of
   (a) obtaining tumour and normal DNA or tumour and normal RNA sequence information for a subject diagnosed with cancer;
   (b) identifying subject-specific MHC I and MHC II restricted tumour neoantigens based on the sequence information obtained in (a);
   (c) determining the sequence of a first peptide, wherein the first peptide is an MHC-I antigen and its sequence comprises a sequence comprising a subject-specific tumour neoantigen identified in step (b);
   (d) determining the sequence of a second peptide, wherein the second peptide is an MHC-II antigen, its sequence comprises a sequence comprising a subject-specific tumour neoantigen identified in step (b) and comprises the sequence of the first peptide;
   (e) preparing the first peptide or a nucleic acid encoding the first peptide that can be expressed upon administration of said nucleic acid to a subject;
   (f) preparing the second peptide or a nucleic acid encoding the second peptide that can be expressed upon administration of said nucleic acid to a subject; and
   (g) formulating the subject-specific vaccine composition using the preparations of (e) and (f).
15. The method of paragraph 14, wherein the first peptide is prepared in a form of a peptide or a pharmaceutically acceptable salt thereof, and/or
   wherein the second peptide is prepared in a form of a peptide or a pharmaceutically acceptable salt thereof, and/or
   wherein the sequence of the first peptide is 8 to 13 amino acid residues long, and/or
   wherein the sequence of the second peptide is at least 14 amino acid residues long, preferably 14 to 35 amino acid residues long, and/or
   wherein the first peptide is predicted to be an MHC-I antigen by using a bioinformatic prediction algorithm, and/or
   wherein the second peptide is predicted to be an MHC-II antigen by using a bioinformatic prediction algorithm.

1. A vaccine composition comprising
a first peptide or a nucleic acid encoding the first peptide that can be expressed upon administration of said nucleic acid to a subject, wherein the first peptide is an MHC I antigen; and
a second peptide or a nucleic acid encoding the second peptide that can be expressed upon administration of said nucleic acid to a subject, wherein the second peptide is an MHC II antigen;
wherein the sequence of the first peptide is comprised in the sequence of the second peptide.

2. A kit of parts comprising
a vaccine composition comprising a first peptide or a nucleic acid encoding the first peptide that can be expressed upon administration of said nucleic acid to a subject, wherein the first peptide is an MHC I antigen; and
a vaccine composition comprising a second peptide or a nucleic acid encoding the second peptide that can be expressed upon administration of said nucleic acid to a subject, wherein the second peptide is an MHC II antigen;
wherein the sequence of the first peptide is comprised in the sequence of the second peptide.

3. The vaccine composition of claim 1, wherein the first peptide is present in a form of a peptide or a pharmaceutically acceptable salt thereof.

4. The vaccine composition of claim 1, wherein the first peptide is present in a form of a nucleic acid encoding the first peptide that can be expressed upon administration of said nucleic acid to a subject.

5. The vaccine composition of claim 1, wherein the second peptide is present in a form of a peptide or a pharmaceutically acceptable salt thereof.

6. The vaccine composition of claim 1, wherein the second peptide is present in a form of a nucleic acid encoding the second peptide that can be expressed upon administration of said nucleic acid to a subject.

7. The vaccine composition of claim 1, wherein the sequence of the first peptide is 8 to 13 amino acid residues long.

8. The vaccine composition of claim 1, wherein the sequence of the second peptide is at least 14 amino acid residues long, preferably is 14 to 35 amino acid residues long.

9. The vaccine composition of claim 1, wherein the first peptide is determined to be an MHC I antigen by using one or more bioinformatic epitope prediction algorithms, preferably selected from NetMHC, NetMHCpan, MHCflurry, Puffin, SYFPEITHI, and MixMHCpred.

10. The vaccine composition of claim 1, wherein the second peptide is determined to be an MHC II antigen by using one or more bioinformatic epitope prediction algorithms, preferably selected from NetMHCII, NetMHCIIpan, MixMHC2pred, MARIA, SYFPEITHI, TEPITOPE, SMM-Align, BERTMHC and Multipred.

11. The vaccine composition of claim 1, wherein the sequence of the second peptide comprises more than one sequence of an MHC II antigen, preferably five or more sequences of an HMC I antigen, preferably as determined using one or more bioinformatic prediction algorithms.

12. The vaccine composition of claim 1, wherein the composition comprises more than one first peptide which are configured to be different MHC I antigens and wherein the sequence of each of said first peptides is comprised in the sequence of the second peptide which is configured to be an MHC II antigen.

13. (canceled)

14. The vaccine composition of claim 1, wherein the sequence of the first peptide is derived from a tumour neoantigen.

15. The vaccine composition of claim 1, wherein the sequence of the first peptide comprises a cancer-specific non-synonymous mutation, preferably a subject specific and tumour-specific mutation.

16. The vaccine composition of claim 14, wherein the vaccine composition is tailored to a specific subject by the sequence of the first peptide:
being a sequence derived from a subject-specific tumour neoantigen;
comprising a subject-specific tumour mutation; and/or
being a sequence of an unmutated tumour associated antigen.

17-18. (canceled)

19. The vaccine composition of claim 1, wherein the vaccine composition is an anti-pathogen vaccine composition or wherein the vaccine compositions are anti-pathogen vaccine compositions, wherein the sequence of the first peptide is derived from a pathogen, preferably a virus, a bacterium, or a fungus.

20-25. (canceled)

26. A method for preparing a subject-specific vaccine composition for use in cancer therapy, the method comprising the steps of
(a) obtaining tumour and normal DNA or tumour and normal RNA sequence information for a subject diagnosed with cancer;
(b) identifying subject-specific tumour-mutations and subject-specific MHC I and MHC II restricted tumour neoantigens based on the sequence information obtained in (a);
(c) determining the sequence of a first peptide, wherein the first peptide is an MHC I antigen and its sequence comprises a sequence comprising a subject-specific tumour neoantigen identified in step (b);
(d) determining the sequence of a corresponding second peptide, wherein the second peptide is an MHC II antigen, its sequence comprises a sequence comprising a subject-specific tumour neoantigen identified in step (b) and comprises the sequence of the first peptide;
(e) preparing the first peptide or a nucleic acid encoding the first peptide that can be expressed upon administration of said nucleic acid to a subject;
(f) preparing the second peptide or a nucleic acid encoding the second peptide that can be expressed upon administration of said nucleic acid to a subject; and
(g) formulating the subject-specific vaccine composition using the preparations of (e) and (f).

27-35. (canceled)

36. A method of inducing an immune response in a subject, the method comprising administering the vaccine composition of claim 1 to a subject in need thereof.

37. A method of increasing an immune response in a subject, the method comprising administering the vaccine composition of claim 1 to a subject in need thereof.

38. A method of treating a cancer disease in a subject, the method comprising administering the vaccine composition of claim 1 to a subject in need thereof.

39. A method of treating or preventing an infectious disease in a subject, preferably a viral infectious disease, a bacterial infectious disease or a fungal infectious disease, the method comprising administering the vaccine composition of claim 19 to a subject in need thereof.

40-43. (canceled)
